(19) United States
(12) Patent Application Publication
(10) Pub. No.: US 2010/0190842 A1
(43) Pub. Date: Jul. 29, 2010

(54) INFLUENZA POLYNUCLEOTIDES, EXPRESSION CONSTRUCTS, COMPOSITIONS, AND METHODS OF USE

(75) Inventors: Catherine J. Pachuk, Cambridge, MA (US); Daniel E. McCallus, Cambridge, MA (US)

Correspondence Address: David S. Resnick, Nixon Peabody LLP, 100 Summer Street, Boston, MA, 02110, US

(73) Assignee: ALNYLAM PHARMACEUTICALS, INC., Cambridge, MA (US)

(21) Appl. No.: 12/595,412

(22) PCT Filed: Apr. 14, 2008
(86) PCT No.: PCT/US08/60222

§ 371 Date: Apr. 6, 2010

Related U.S. Application Data
(60) Provisional application No. 60/907,650, filed on Apr. 12, 2007.

(51) Int. Cl. A61K 31/7088; C07H 21/04; A61P 31/16; C12N 15/63

(52) U.S. Cl. 514/44 R; 536/23.1; 435/320.1

(57) ABSTRACT

The invention provides isolated RNA molecules containing a stretch of nucleotides from a conserved Influenza sequence, and provides complementary RNA molecules thereto. The RNA molecules of the invention are also substantially non-homologous to human sequences. The RNA molecules of the invention include double-stranded RNAs comprising a first region that is a conserved Influenza sequence, and a second region that is at least substantially complementary to the first region. Such double-stranded RNAs include single short hairpin RNAs (shRNAs) as well as multi-target hairpin RNAs containing a plurality, or several, stem-loop structures. The present invention further provides expression constructs that provide for expression of one, or a plurality, of RNA molecules of the invention. The RNA molecules, expression constructs, and compositions of the present invention find use in reducing levels of Influenza A RNA, in reducing Influenza A virus titer, and in treating or preventing Influenza virus infection. The invention is effective against at least human, swine and avian originating strains of Influenza A, and makes gene-silencing therapeutic strategies for combating Influenza A infection feasible.

### CROSS REFERENCE TO RELATED APPLICATIONS

This application claims priority to U.S. Provisional Application No. 60/907,650, filed Apr. 12, 2007, which is herein incorporated by reference in its entirety.

### FIELD OF THE INVENTION

The present invention relates to nucleic acid-based therapeutics for treating or preventing Influenza replication and/or infection, such as RNAi-based therapeutics.

### BACKGROUND OF THE INVENTION

Influenza is an acute respiratory illness of global significance. Despite international attempts to control influenza virus outbreaks through vaccination, influenza infections remain a major cause of morbidity and mortality. Worldwide influenza pandemics have occurred irregularly and unpredictably throughout history, and it is expected that these sporadic pandemics will continue.

While vaccination remains the most effective defense against influenza virus, its effectiveness is limited by the influenza virus' constant mutation to accommodate environmental change. In fact, the only influenza epitopes known to elicit strong humoral responses are non-conserved, which requires that new vaccines be developed continually. New strategies for treatment and/or prevention of influenza virus infections are therefore critical for improving human and animal health world wide.

The ability of double-stranded RNA to effectively silence gene expression is a phenomenon commonly known as RNA interference (RNAi), and efforts are underway to apply RNAi for the treatment of human disease. RNA interference refers to the process of sequence-specific post-transcriptional gene silencing in animals mediated by short interfering RNAs (siRNAs). Briefly, the presence of dsRNA in cells can stimulate the activity of a ribonuclease III enzyme referred to as dicer (Bass, 2000, Cell, 101, 235; Zamore et al., 2000, Cell, 101, 25 33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25 33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25 33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

However, to apply such a gene silencing strategy to the treatment or prevention of influenza virus infection, it is necessary to identify sufficiently conserved stretches of nucleotide sequence in this highly mutable virus. That is, since RNA interference is a sequence-specific effect, the therapeutic or prophylactic RNAi molecules must be specific for influenza target sequences, despite the fact that influenza viral genomes are highly variable.

In addition to being specific for conserved influenza target sequences, such RNAi molecules must also be substantially non-homologous to naturally occurring, normally functioning, host polynucleotide sequences, so that the therapeutic or prophylactic strategy does not adversely affect the function of any essential host gene.

### SUMMARY OF INVENTION

The eight Influenza A genomic RNA segments were compared from 16,015 Influenza A virus sequences. These Influenza A sequences were from 20 different subtypes and twelve different hosts including human, avian, swine, equine, and mouse. Fourteen conserved stretches of greater than 21 nucleotides in length were identified.

The invention provides polynucleotides, including RNA molecules, containing a stretch of nucleotides from a conserved Influenza sequence. The polynucleotides of the invention are also substantially non-homologous to human sequences. The present invention further provides polynucleotides containing a stretch of nucleotides complementary to, or substantially complementary to, a conserved Influenza sequence.

The polynucleotides of the invention include double-stranded RNAs comprising a first region or strand that is a conserved Influenza sequence, and a second region or strand that is at least substantially complementary to the first. Such double-stranded RNAs include dsRNA complexes, single short hairpin RNAs (shRNAs) as well as multi-target hairpin RNAs containing a plurality, or several, stem-loop structures containing conserved Influenza sequences.

The present invention further provides expression constructs that provide for expression of one, or a plurality, of RNA molecules of the invention.

The present invention provides compositions comprising one, or two or more, RNA molecules of the invention, or alternatively expression construct(s) of the invention, together with a pharmaceutically acceptable carrier.

The polynucleotides, expression constructs, and compositions of the present invention find use in preventing Influenza A replication in a cell, prohibiting or reducing levels of Influenza A RNA in a cell, reducing Influenza A virus titer, and treating or preventing Influenza virus infection, as well as other uses. The invention is effective against at least human, swine and avian originating strains of Influenza A, and thereby makes gene-silencing prophylactic and therapeutic strategies for combating Influenza A infection and transmission feasible. The present invention finds therapeutic and prophylactic use from season-to-season, unlike Influenza vaccine strategies, which are hampered by rapidly changing antigenic epitopes.

### BRIEF DESCRIPTION OF THE DRAWINGS

FIG. 1 depicts exemplary shRNA and siRNA sequences derived from different conserved regions of various segments of the influenza A viral genome. The bolded sequences represent consensus sequences from a particular conserved region of the specified segment. The shRNA sequences shown on the left-hand side of the table comprise, in a 5′ to 3′ direction: an antisense sequence; a single-stranded loop sequence (underlined); and a sense sequence, wherein the sense sequence is the siRNA sequence depicted in the right-most column of the table. The size of the siRNA sequences (sense sequences) are listed in the far left column (e.g. 21-mers, 25-mers, and 27-mers).

FIG. 2 shows additional siRNA sequences designed from conserved regions of influenza A virus segments 1, 5, 7, and 8. Nucleotide positions refer to specific nucleotide ranges in accordance with GenBank Accession Nos. V00603 (segment 1), V01084 (segment 5), NC_{—}002016 (segment 7), and J02150 (segment 8). The nucleotide sequences of GenBank Accession Nos.: V00603, V01084, NC_{—}002016, and J02150 are hereby incorporated by reference.

FIG. 3 depicts the results of hemagglutination assays of MDCK cells that were transfected with either a control plasmid (Nuc067) or a plasmid expressing a shRNA directed toward the PB2 gene of influenza A virus (3.21.11) (SEQ ID NO: 197). “Mock” treatment denotes cells that went through the transfection procedure but were not transfected with plasmid DNA.

### DETAILED DESCRIPTION OF INVENTION

The eight Influenza A RNA segments were compared from 16,015 Influenza A virus sequences. These Influenza A sequences were from 200 different subtypes and twelve different hosts including human, avian, swine, equine, and mouse (see Table 1). Fourteen conserved regions of greater than 21 nucleotides in length were identified (see Tables 2-15), which represent appropriate targets for gene silencing, including RNAi-based gene silencing.

These conserved regions were further screened against Genbank sequences (Human, Mouse, and Rat cDNA sequence databases), to identify and eliminate any Influenza sequences (from the fourteen conserved regions) that could potentially interfere with host cellular functions. Several highly conserved Influenza A sequences were identified, which are not only highly conserved across human, avian and swine Influenza strains, but are also substantially non-homologous to human sequences.

The term “conserved” or “highly conserved” according to this invention means that the stretch of nucleic acids is sufficiently conserved to act as a target for RNAi based therapeutics. For example, a conserved Influenza sequence may be variable at from 1 to about 5 nucleotides, in a stretch of at least about 19 nucleotides, such as from about 21 to about 29 nucleotides. In some embodiments, the conserved sequence is variable at only 1, 2, or 3 positions. Further, in some embodiments, a variable nucleotide is limited to either a purine or pyrimidine nucleotide.

### Polynucleotides

The invention provides polynucleotides, such as RNA molecules, containing a stretch of nucleotides from a conserved Influenza A sequence, or complementary to a conserved Influenza sequence, such as from one of conserved regions 1-14 as described herein. For example, the RNA molecule may contain a stretch of nucleotides from one or more of Conserved Regions 3, 4, 5, 6, 12, and/or 14, and/or a stretch of nucleotides complementary thereto.

In one embodiment, the invention provides an isolated RNA molecule containing 19 or more contiguous nucleotides of a sequence selected from:

(SEQ ID NO: 1)

GGGCAAGGAGACGURGUGUUGGUAAUGAAACG,

(SEQ ID NO: 2)

GACAACAUGACCAAGAAAAUGGUCACACAAAGAACAAUAGG,

(SEQ ID NO: 3)

CGYAGGCUUGCCGACCAAAGUCUCCC,

(SEQ ID NO: 4)

UUUAGAGCCUAUGUGGAUGGAUU,

(SEQ ID NO: 5)

AUGGCGUCYCAAGGCACCAAACGRUCUUAUGARCA,

(SEQ ID NO: 6)

AGGCCCCCUCAAAGCCGARAUCGCDCAGAVACUUGAA,

(SEQ ID NO: 7)

UUUGURUUCACGCUCACCGUGCCCAGUGAGCGR,

(SEQ ID NO: 8)

AGAUGAUCUUCUUGAAAAUUUGCAGVCCUAYCAGAAACGRAUGGG,
and

(SEQ ID NO: 9)

GAGGAUGUCAAAAAUGCAAUUGGGGUCCUCAUCVDAGGRHUUGAA
UGGA.

As used herein, a nucleotide designated as R is A or G; a nucleotide designated as Y is U or C; a nucleotide designated as D is A, G, or U; a nucleotide designated as V is A, G, or C; and a nucleotide designated as H is C, A, or U. In certain embodiments, the RNA molecule of the invention includes no more than one nucleotide designated as R, Y, D, V, or H in SEQ ID NOS: 1-9.

In certain embodiments of the invention, the RNA molecule contains 19 or more contiguous nucleotides of a sequence selected from:

(SEQ ID NO: 10)

GGGCAAGGAGACGUGGUGUUGGUAAUGAAACG,

(SEQ ID NO: 11)

GGGCAAGGAGACGUGAUGUUGGUAAUGAAACG,

(SEQ ID NO: 12)

CGCAGGCUUGCCGACCAAAGUCUCCC,

(SEQ ID NO: 13)

CGUAGGCUUGCCGACCAAAGUCUCCC,

(SEQ ID NO: 14)

AUGGCGUCUCAAGGCACCAAACG,

(SEQ ID NO: 15)

AUGGCGUCCCAAGGCACCAAACG,

(SEQ ID NO: 16)

AGGCCCCCUCAAAGCCGAGAUCGC,

(SEQ ID NO: 17)

AGGCCCCCUCAAAGCCGAAAUCGC,

(SEQ ID NO: 18)

UUUGUAUUCACGCUCACCGUGCCCAGUGAGCGA,

(SEQ ID NO: 19)

UUUGUGUUCACGCUCACCGUGCCCAGUGAGCG,

(SEQ ID NO: 20)

UUCACGCUCACCGUGCCCAGUGAGCG,

(SEQ ID NO: 21)

AGAUGAUCUUCUUGAAAAUUUGCAGGCCUA,

(SEQ ID NO: 22)

AGAUGAUCUUCUUGAAAAUUUGCAGACCUA,
and

(SEQ ID NO: 23)

GAGGAUGUCAAAAAUGCAAUUGGGGUCCUCAUCG.

In other embodiments, the RNA molecule contains 19 or more contiguous nucleotides of a sequence selected from SEQ ID NOs: 52-186. The invention further provides DNA molecules corresponding to the RNA molecules of the invention.

In one embodiment, among others, the RNA molecule of the invention is of a length suitable for RNAi-based gene silencing. Thus, for example, the RNA molecule may contain a conserved influenza sequence of from about 19 to about 29 nucleotides in length. In some embodiments, the conserved sequence is from about 20 to about 27 nucleotides in length, or from about 21 to about 25 nucleotides in length. In certain embodiments, the RNA molecule of the invention consists of, or consists essentially of, the Influenza A conserved sequence.

The RNA molecule of the invention targets cellular Influenza RNA sequences by RNAi-based gene silencing when the RNA molecule, or a region of the RNA molecule, is hybridized to a substantially complementary RNA molecule or region. As used herein, the term “substantially complementary” means sufficiently complementary to support RNAi-based gene silencing. Thus, the term “substantially complementary” encompasses complete complementarity between two RNA segments of the same or different sizes, or at least sufficient complementarity to trigger the cellular RNAi machinery. In exemplary embodiments, at least about 19 nucleotides of the RNA of the invention are hybridized to a second RNA segment. In certain embodiments, from about 19 to about 27, or from about 20 to about 26, or from about 21 to about 25, nucleotides are hybridized to the second RNA segment. In these or other embodiments, the RNA molecule of the invention may be linked to the complementary RNA segment, or substantially complementary RNA segment, through for example, a nucleic acid linker. The nucleic acid linker region may be from about 4 to about 30 nucleotides in length, from about 9 to about 15 nucleotides in length, or preferably from about 4 to about 10 nucleotides in length. For example, a single RNA strand may fold back to form a double stranded RNA, where the two complementary portions are optionally separated by a single stranded loop or stuffer region.

The present invention further provides an isolated RNA molecule containing about 19 or more contiguous nucleotides complementary to a sequence selected from SEQ ID NOs: 1-9. In some embodiments, the RNA molecule includes no more than one nucleotide that is complementary to a nucleotide designated as R, Y, D, V, or H in SEQ ID NOs: 1-9. The RNA molecule of the invention, in some embodiments, contains about 19 or more contiguous nucleotides complementary to a sequence selected from SEQ ID NOs: 10-23 and SEQ ID NOs: 52-186.

The RNA molecules having a sequence complementary to one of SEQ ID NOs: 1-9 are also of a length suitable for RNAi-based gene silencing, and thus, the portion complementary to a conserved Influenza sequence may be from about 19 to about 29 nucleotides in length. In some embodiments, the portion complementary to a conserved Influenza sequence is from about 20 to about 27 nucleotides in length, or from about 21 to about 25 nucleotides in length.

The RNA molecule or region complementary to one of SEQ ID NOs: 1-9 targets cellular Influenza RNA sequences by RNAi-based gene silencing when the RNA molecule or region is hybridized to a substantially complementary RNA molecule or region. Thus, in exemplary embodiments, at least about 19 nucleotides of the RNA are hybridized to a second RNA segment. In certain embodiments, from about 19 to about 27, or from about 20 to about 26, or from about 21 to about 25 nucleotides are hybridized to the second RNA segment. In these or other embodiments, the RNA molecule of the invention may be linked to the complementary RNA segment, or substantially complementary RNA segment, through for example, a nucleic acid linker. Alternatively, a single RNA strand may fold back to form a double stranded RNA, where the two complementary portions are optionally separated by a single stranded loop or stuffer region.

The invention contemplates the use of polynucleotides comprising naturally occurring nucleotides, as well as polynucleotides containing chemically modified nucleotides. Exemplary chemically modified nucleotides include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These modifications, as well as other chemical modifications, support RNAi-mediated gene silencing, as well as other applications, while having superior serum stability.

In another embodiment, the RNA of the invention is a double-stranded RNA comprising a first region having about 19 or more contiguous nucleotides of a sequence selected from SEQ ID NOs: 1-9, and a second region being at least substantially complementary to the first region. In certain embodiments, the first region of the double-stranded RNA includes no more than one nucleotide designated as R, Y, D, V, or H. The double-stranded RNAs of the invention may have at least 19 nucleotides in double-stranded conformation. In some embodiments, the double-stranded RNA has from about 19 to about 29 nucleotides, or from about 20 to about 27 nucleotides, or from about 21 to about 26 nucleotides, or from about 22 to about 25 nucleotides of one region complementary to another region.

“Double stranded RNA” or “dsRNA” is a ribonucleic acid containing at least a region of nucleotides in a double stranded conformation. The double stranded RNA may be two separate strands, wherein one strand contains a sense sequence and the other strand contains an antisense sequence such that the two strands are capable of hybridizing under physiological conditions to form a duplex. The double stranded RNA may be a single molecule with a region of self-complementarity such that nucleotides in one segment of the molecule base pair with nucleotides in another segment of the molecule. In some embodiments, the double stranded RNA is a single molecule, and/or is composed entirely of ribonucleotides. The invention further contemplates the use of RNA molecules that include a region of ribonucleotides that is complementary to a region of deoxyribonucleotides. Alternatively, the double stranded RNA may include two different strands that have a region of complementarity to each other. Preferably, the double stranded RNA includes at least about 15, 20, 25, 30, 50, 75, 100, or 200 nucleotides in double-stranded conformation. In some embodiments, the double-stranded RNA is fully complementary, and does not contain any single stranded regions, such as single stranded ends. In other embodiments, the dsRNA contains short single-stranded ends, such as single-stranded 3′ ends of from about 1 to about 5 nucleotides (e.g., 1, 2, 3, or 4 nucleotides).

Generally, the double stranded region(s) of the RNA molecule correspond to one or more Influenza target sequence(s), for instance, for mediating RNA interference. In such instances, the dsRNA region(s) are substantially homologous and complementary to a region of a target sequence. Where the dsRNA is used for RNA interference, one strand of the dsRNA structure or region, i.e., the antisense strand, will have at least about 70, 80, 90, 95, 98, or 100% complementarity to a target nucleic acid, and the other strand or region, i.e., the sense strand or region will have at least about 70, 80, 90, 95, 98, or 100% identity to a target nucleic acid. In such embodiments, the dsRNA is considered to be both substantially homologous and complementary to the target sequence, meaning that the dsRNA need not be entirely identical and complementary to the target sequence so long as it is still effective to mediate sequence-specific RNA interference.

In one embodiment, the dsRNA is a short hairpin dsRNA (shRNA) or a microRNA. A “shRNA” (short-hairpin RNA) is an RNA molecule of less than approximately 500 or 400 nucleotides, and preferably less than about 200 or about 100 nucleotides, in which at least one stretch of nucleotides (e.g., at least about 19 nucleotides) is base paired with a complementary sequence located on the same RNA molecule and separated from the complementary sequence by an unpaired region of at least about 4 nucleotides, such as about 9 nucleotides. These single-stranded hairpin regions form a single-stranded loop between the stem structure created by the two regions of base complementarity. The single-stranded hairpin region or loop region may be from about 4 to about 30 nucleotides in length, from about 9 to about 15 nucleotides in length, or preferably about 4 to about 10 nucleotides in length.

In some embodiments, the shRNAs may comprise in 5′ to 3′ order: a sequence that is substantially complementary to one of the Influenza target sequences disclosed herein (antisense), a single-stranded loop or hairpin region, and a sequence that is substantially identical to one of the Influenza target sequences disclosed herein (sense). In other embodiments, the shRNAs may comprise in 5′ to 3′ order: a sense sequence that is substantially identical to a target sequence disclosed herein, a single-stranded loop or hairpin region, and an antisense sequence that is substantially complementary to a target sequence disclosed herein. In preferred embodiments, the shRNAs may contain a sequence selected from SEQ ID NOs: 187-268.

In addition to single shRNAs, the invention includes dual or bi-finger and multi-finger hairpin dsRNAs, in which the RNA molecule comprises two or more of such stem-loop structures each separated by a single-stranded spacer region. The hairpin dsRNA may be a single hairpin dsRNA or a bi-fingered, or multi-fingered dsRNA hairpin as described in PCT/US03/033466 or WO 04/035766, or a partial or forced hairpin structure as described in WO 2004/011624, the teachings of which are incorporated herein by reference in their entireties.

The length of the double stranded RNAs of the invention, or the length of the double stranded regions, is such that the double-stranded RNA is able to trigger RNAi-mediated degradation of the target Influenza sequence(s). Thus, the first region and/or the second region (the complementary region) of the double stranded RNA may be from about 19 to about 26 nucleotides in length. While the double stranded RNA of the invention may exist in a denatured or substantially denatured form, the invention contemplates molecules in a double stranded conformation, or a substantially double stranded conformation, or a partially double-stranded conformation.

In certain embodiments, the dsRNA is a multi-target double-stranded RNA comprising two or more segments each consisting of about 19 or more contiguous nucleotides of a sequence independently selected from SEQ ID NOs: 1-9, and a substantially complementary region for each segment. According to this embodiment, each of the two or more segments is connected to its complementary region through a single-stranded loop or stuffer region. Each segment and substantially complementary region of the multi-target double-stranded RNA is capable of triggering RNAi-mediated degradation of a target Influenza sequence. For example, in certain embodiments, each complementary region contains at least 19 complementary nucleotides (e.g., nucleotides complementary to the corresponding segment of the multi-target double-stranded RNA). Thus, the invention contemplates embodiments in which each complementary region contains from about 19 to about 29 complementary nucleotides, or from about 20 to about 27 complementary nucleotides, or from about 21 to about 26 complementary nucleotides, or from about 22 to about 25 complementary nucleotides.

The multi-target double stranded RNA may contain double-stranded regions sufficient to trigger RNAi mediated gene-silencing of one or more of Influenza A Conserved Regions 1-14, as described herein. For example, the multi-target dsRNA may target one or more of Conserved Regions 3, 4, 5, 6, 12, and/or 14. In one embodiment, the multi-target double stranded RNA contains Influenza A sequences from one or more of Conserved Region 3, Conserved Region 5, Conserved Region 6 and Conserved Region 12, as described more fully herein.

### Constructs

In another aspect, the present invention provides an expression construct containing a DNA segment that encodes an RNA molecule of the invention, with the DNA segment being operably linked to a promoter to drive expression of the RNA molecule. It is understood that DNA sequences corresponding to or encoding one or more of the RNA sequences disclosed herein would contain a thymidine (T) base instead of a uracil (U) base. An “expression construct” is any double-stranded DNA or double-stranded RNA designed to produce an RNA of interest. For example, the construct contains at least one promoter that is, or may be, operably linked to a downstream gene, coding region, or polynucleotide sequence of interest. A polynucleotide sequence of interest may be: a cDNA or genomic DNA fragment, either protein encoding or non-encoding; an RNA effector molecule such as an antisense RNA, triplex-forming RNA, ribozyme, an artificially selected high affinity RNA ligand (aptamer); a double-stranded RNA, e.g., an RNA molecule comprising a stem-loop or hairpin dsRNA, or a bi-finger or multi-finger dsRNA or a microRNA, or any RNA of interest. The invention includes expression constructs in which one or more of the promoters is not in fact operably linked to a polynucleotide sequence to be transcribed, but instead is designed for efficient insertion of an operably-linked polynucleotide sequence to be transcribed by the promoter, for instance by way of one or more restriction cloning sites in operative association with the one or more promoters.

Transfection or transformation of the expression construct into a recipient cell allows the cell to express an RNA effector molecule encoded by the expression construct. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus. Expression vectors for use with the invention contain sequences from bacteria, viruses or phages. Such vectors include chromosomal, episomal and virus-derived vectors, e.g., vectors derived from bacterial plasmids, bacteriophages, yeast episomes, yeast chromosomal elements, and viruses; as well as vectors derived from combinations thereof, such as those derived from plasmid and bacteriophage genetic elements, cosmids and phagemids. Exemplary vectors are double-stranded DNA phage vectors and double-stranded DNA viral vectors.

An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the expression construct of the invention is a plasmid.

An expression construct may be engineered to encode multiple, e.g., three, four, five or more RNA molecules, such as short hairpin dsRNAs and/or other RNAs. The encoded RNAs may be separate, or in the form of bi-finger or multi-finger constructs comprising hairpin or stem loop regions according to the invention separated by a single-stranded region of at least about 5, 10, 15, 20, or 25 nucleotides or more. See application nos. WO 2000/63364 and WO 2004/035765, which are hereby incorporated by reference in their entireties.

In addition to utilizing highly conserved sequences, the ability to co-deliver two, three, four, five or more different RNA effector molecules radically reduces the ability of the virus to develop escape mutants. While “cocktail” pharmaceutical preparations including multiple active components can be formulated, dsRNA expression constructs provide an attractive delivery vehicle for accomplishing such co-delivery of a plurality of different antiviral effector molecules.

In certain embodiments, the expression construct encodes two or more RNAs of the invention, such as 2, 3, 4, 5, or more double stranded RNA molecules, such as duplexes comprising two separate strands. The construct may further encode double-stranded RNAs as double-stranded hairpin molecules. Thus, in one embodiment, the expression construct encodes from 2, 3, 4, 5, or more dsRNA hairpins. For example, the expression construct may encode double stranded RNAs, such as dsRNA hairpins or duplexes, specific for one or more of Conserved Regions 1-14, such as Conserved Region 3, Conserved Region 5, Conserved Region 6 and Conserved Region 12. Alternatively, the dsRNA hairpins may be combined into one or a plurality of multi-target double-stranded RNAs.

Where it is desired to deliver short dsRNAs, multiple RNA polymerase III promoter expression constructs (as taught in WO 06/033756, which is hereby incorporated by reference in its entirety), may be used in accordance with the invention. The multiple RNA polymerase III promoters may be utilized in conjunction with promoters of other classes, including RNA polymerase I promoters, RNA polymerase II promoters, etc. Preferred in some applications are the Type III RNA pol III promoters including U6, H1, and 7SK, which exist in the 5′ flanking region, include TATA boxes, and lack internal promoter sequences. A preferred 7SK promoter is the 7SK 4A promoter variant taught in WO 06/033756, the nucleotide sequence of which is hereby incorporated by reference. In such expression constructs each promoter may be designed to control expression of an independent RNA expression cassette, e.g., a shRNA expression cassette. In some embodiments where production of a double stranded duplex is desired, one promoter may control the expression of the sense strand, while a second promoter controls the expression of the antisense strand. The two promoters may be located on the same vector molecule or on separate vector molecules. RNA Pol III promoters may be especially beneficial for expression of small engineered RNA transcripts, because RNA Pol III termination occurs efficiently and precisely at a short run of thymine residues in the DNA coding strand, without other protein factors. T₄ and T₅ are the shortest Pol III termination signals in yeast and mammals, with oligo (dT) terminators longer than T₅ being rare in mammals. Accordingly, the multiple polymerase III promoter expression constructs of the invention will include an appropriate oligo (dT) termination signal, i.e., a sequence of 4, 5, 6 or more Ts, operably linked 3′ to each RNA Pol III promoter in the DNA coding strand. A DNA sequence encoding an RNA effector molecule, e.g., a dsRNA hairpin or RNA stem-loop structure to be transcribed, is inserted between the Pol III promoter and the termination signal.

The invention provides means for delivering to a host cell sustained amounts of 2, 3, 4, 5, or more different antiviral dsRNA hairpin molecules (e.g., specific for 2, 3, 4, 5, or more different viral sequence elements), in a genetically stable mode, so as to inhibit viral replication without evoking a dsRNA stress response. In accordance with this aspect, each dsRNA hairpin may be expressed from an expression construct, and controlled by an RNA polymerase III promoter.

Thus, the expression constructs of the invention provide a convenient means for delivering a multi-drug regimen comprising several different RNAs of the invention to a cell or tissue of a host vertebrate organism, thereby potentiating the anti-viral activity, and reducing the likelihood that multiple independent mutational events will produce resistant virus. This provides an important advantage in countering viral variation both within human and animal host populations and temporally within a host due to mutation events.

Another aspect of the invention is a composition comprising two or more RNAs, each containing 19 or more contiguous nucleotides of a sequence selected from SEQ ID NOs: 1-9, and the composition further comprising the substantially complementary RNA molecule or region for each of said two or more RNAs. The composition of the invention may contain a pharmaceutically acceptable carrier. The invention also provides a composition comprising an expression construct encoding at least two RNA molecules of the invention, and a pharmaceutically acceptable carrier. In certain embodiments, the composition is formulated for administration by injection or inhalation.

The compositions of the invention include RNAs that are chemically stabilized and/or chemically modified, using one or more of the methods and chemical modifications known to those of skill in the art.

In various embodiments, the pharmaceutical composition includes about 1 ng to about 20 mg of nucleic acid, e.g., RNA, DNA, plasmids, viral vectors, recombinant viruses, or mixtures thereof, which provide the desired amounts of the nucleic acid molecules. In some embodiments, the composition contains about 10 ng to about 10 mg of nucleic acid, about 0.1 mg to about 500 mg, about 1 mg to about 350 mg, about 25 mg to about 250 mg, or about 100 mg of nucleic acid. Those of skill in the art of clinical pharmacology can readily arrive at such dosing schedules using routine experimentation.

Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid. In some embodiments, the pharmaceutical composition contains one or more pharmaceutically acceptable additives suitable for the selected route and mode of administration. These compositions may be administered by, without limitation, any parenteral route including intravenous (IV), intra-arterial, intramuscular (IM), subcutaneous (SC), intradermal, intraperitoneal, intrathecal, as well as topically, orally, and by mucosal routes of delivery such as intranasal, inhalation, rectal, vaginal, buccal, and sublingual. Preferably, the compositions are administered by inhalation. In some embodiments, the pharmaceutical compositions of the invention are prepared for administration to a vertebrate subject (e.g., mammalian subjects including human, canine, feline, bovine, equine, porcine; as well as avian subjects such as poultry) in the form of liquids, including sterile, non-pyrogenic liquids for injection, emulsions, powders, aerosols, tablets, capsules, enteric coated tablets, or suppositories.

The compounds and compositions of the invention may be prepared using conventional techniques well known in the art.

### Methods of Prophylaxis and Treatment

The present invention provides numerous methods of using the RNA molecules, expression constructs and compositions of the invention.

For example, the compounds and compositions of the invention find use in prophylaxis of Influenza virus replication in a cell, and of Influenza virus infection of a cell or host. While seasonal vaccination is available for prevention of flu, vaccine induced protection is mediated by neutralizing humoral immune responses to antigenic peptide epitopes on the neuraminidase and/or the hemagglutinin protein(s) displayed on the surface of the influenza viral particle. Due to the high level of variation in these epitope sequences amongst viral isolates and strains, and because strains responsible for seasonal flu outbreaks change year to year, a different vaccine must be generated each year. Effectiveness of the vaccine is variable, in part due to inaccuracies in predicting the influenza A strains of the upcoming flu season and in part due to ineffectiveness in the population. According to the CDC, in years where the vaccine is well matched to the circulating strains, effectiveness occurs in 70-90% of the adults who are under 65 years of age. Effectiveness is less in the juvenile and elderly populations. In years when the vaccine is not well matched to circulating strains, effectiveness drops to about 55% in the adult population.

Further, flu vaccines are not 100% effective, in part, due to error in prediction of seasonal flu strains and in part due to decreased response rates in certain segments of the population. Vaccine composition changes from season to season, making manufacturing and stockpiling difficult, while drug treatments are ineffective in normal individuals due to the need to treat early in the infection cycle.

The compounds and compositions of the invention further find use as therapeutics for Influenza virus replication in a cell, and for Influenza virus infection of a cell or host. While therapeutics such as Tamiflu exist for the treatment of flu, these have only marginal activity and must be administered shortly after the first symptoms of flu occur. This is because peak flu replication in healthy adults has been shown to occur before the occurrence of symptoms, which first appear when interferon is produced concomitant with the decline of viral replication. Many symptoms of the flu are mediated by effects of interferon and other cytokines that are released and are not directly attributable to replicating virus. Additionally, while Tamiflu has a modest effect on improving recovery time from influenza it has been shown to be much less effective for avian flu.

In various embodiments, the compounds, compositions, and methods of the invention are effective season to season against all Influenza A strains, including avian influenza. This is because, unlike vaccines, which are based on non-conserved and highly variable viral protein sequences, the present invention is based on highly conserved influenza RNA sequences. Further, the use of multiple conserved sequences in a single product allows for the development of a product that is active against most influenza viruses, including avian influenza, and can be used year to year for seasonal outbreaks including outbreaks with pandemic strains.

The compounds and compositions of the invention are useful for the prevention of influenza. Since influenza infects bronchial epithelial cells in the upper airway, the compounds and compositions of the invention are preferably administered by inhalation, sufficient to enable transfection of bronchial epithelial cells, for example, with an eiRNA-based plasmid of the invention. The methods of the invention, in some embodiments, obtain persistence of both eiRNA plasmid and persistence of expression for the lifetime of the transfected cell. Transfected plasmid DNA is eventually lost through cell turnover and cell division. Bronchial epithelial cells have been shown to turn over at a rate of about 1% per day and therefore, the half-life of activity may be around 50 days. Thus, in accordance with the methods of the invention, the compounds and compositions of the invention are preferably administered about twice during flu season, or about once every two months.

The compounds and compositions of the invention may target multiple conserved sequences that encompass several different viral chromosomes (or segments). Influenza mRNA, cRNA and vRNA synthesis is impaired as is translation of proteins from the targeted mRNAs. Replication of the virus is therefore also severely impacted. Cells harboring the products are expected to be resistant to direct infection by the virus from contagion as well as resistant to cell-to-cell spread of infectious virus from neighboring infected cells.

For example, one aspect of the invention is a method of preventing influenza replication (e.g., Influenza A) or reducing levels of Influenza A RNA in a cell either in vitro or in vivo. This method comprises introducing a double-stranded RNA of the invention, or a composition of the invention, into a host cell susceptible to Influenza A infection. The method of the invention is effective for human, swine and avian originating strains of Influenza A virus. In accordance with this aspect, the double-stranded RNA may be introduced into the cell by transforming or transfecting the cell, or another cell of an infected organism or tissue, with an expression construct of the invention. Alternatively, the dsRNA may be introduced directly into the cell.

In another aspect, the invention provides a method for preventing or treating Influenza A virus infection of a host or a host cell, or reducing an Influenza A virus titer. This aspect of the invention may also be performed in vitro or in vivo. The method comprises introducing a double-stranded RNA of the invention, or a composition of the invention, into a cell susceptible to Influenza A virus infection. This method is likewise effective against human, swine and avian strains of Influenza A. In accordance with this aspect, the double-stranded RNA may be introduced into the cell by transforming or transfecting a cell with an expression construct of the invention, or alternatively by directly introducing the double stranded RNA.

In another aspect, the invention provides a method of treating a subject having, or at risk of acquiring, an Influenza A viral infection. The method comprises introducing into the subject a double-stranded RNA molecule of the invention, or a composition of the invention. In this aspect, the double-stranded RNA or expression construct directing the production of dsRNA is taken up the host cells, resulting in RNAi-mediated degradation of Influenza A target sequences. This method is effective for human, swine and avian originating strains of Influenza A virus, and may be used in, for example, mammalian or avian subjects, such as human, canine, feline, bovine, equine, and porcine, as well as in poultry.

The invention further provides a use of the compounds and compositions of the invention for the prophylaxis and treatment of, or the manufacture of a medicament for, Influenza A.

In one embodiment of the invention, a double-stranded RNA, or a multi-target double-stranded RNA is introduced into the subject by administering an expression construct providing for expression in the subject of the double-stranded RNA, or the multi-target double-stranded RNA molecule. The term “introducing” a double-stranded RNA includes administering an expression construct in which an RNA molecule and its substantially complementary RNA molecule are expressed separately, that is from separate promoters. In this embodiment, the double-stranded molecule is produced intracellularly upon hybridization of the complementary transcripts.

In one embodiment, the double-stranded RNA molecule, or complementary RNA molecules, are encoded by a single plasmid construct, which may be administered by inhalation.

The method of the invention is suitable for treating or preventing infections of Influenza A virus strains having a human, swine or avian origin, or some combination thereof.

The present invention provides RNA, compositions and methods for modulating levels of Influenza RNA. To “modulate” means to decrease the expression of a target nucleic acid in a cell, or the biological activity of the encoded target polypeptide in a cell, by least 20%, more desirably by at least 30%, 40%, 50%, 60%, 75%, 80%, 85%, 90%, 95% or even 100%. In some instances, expression of genes in the target cell may also be increased, for instance where the gene targeted by the dsRNA is a transcriptional repressor or other negative regulatory gene.

Typically, with expressed interfering RNA (eiRNA), the dsRNA is expressed in the first transfected cell from an expression vector. In such a vector, the sense strand and the antisense strand of the dsRNA may be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Alternatively, the nucleic acid sequence encoding the dsRNA comprises an inverted repeat, such that upon transcription from a single promoter, the expressed RNA forms a double stranded RNA, i.e. that has a hairpin or “stem-loop” structure, e.g., an shRNA. The loop between the inverted repeat regions, or sense and antisense regions, is typically at least four base pairs, but can be at least about 10, at least about 15, at least about 20, at least about 25, at least about 30, at least about 50, or at least about 75, or more, or any size that permits formation of the double stranded structure. Multiple stem-loop structures may be formed from a single RNA transcript to generate a multi-target dsRNA. See WO 00/63364, and WO2004/035765, which are herein incorporated by reference in their entireties. Hairpin structures may be partial or forced hairpin structures as described in WO2004/011624, which is incorporated herein by reference.

Some dsRNA sequences, possibly in certain cell types and through certain delivery methods, may result in an interferon response. The methods of the invention may be performed so as not to trigger an interferon/PKR response, for instance by using shorter dsRNA molecules between 20 to 25 base pairs, by expressing dsRNA molecules intracellularly, or by using other methods known in the art. See US Published Application 20040152117, which is herein incorporated by reference in its entirety. For instance, one of the components of an interferon response is the induction of the interferon-induced protein kinase PKR. To prevent an interferon response, interferon and PKR responses may be silenced in the transfected and target cells using a dsRNA species directed against the mRNAs that encode proteins involved in the response. Alternatively, interferon response promoters are silenced using dsRNA, or the expression of proteins or transcription factors that bind interferon response element (IRE) sequences is abolished using dsRNA or other known techniques.

By “under conditions that inhibit or prevent an interferon response or a dsRNA stress response” is meant conditions that prevent or inhibit one or more interferon responses or cellular RNA stress responses involving cell toxicity, cell death, an anti-proliferative response, or a decreased ability of a dsRNA to carry out a PTGS event. These responses include, but are not limited to, interferon induction (both Type 1 and Type II), induction of one or more interferon stimulated genes, PKR activation, 2′5′-OAS activation, and any downstream cellular and/or organismal sequelae that result from the activation/induction of one or more of these responses. By “organismal sequelae” is meant any effect(s) in a whole animal, organ, or more locally (e.g., at a site of injection) caused by the stress response. Exemplary manifestations include elevated cytokine production, local inflammation, and necrosis. Desirably the conditions that inhibit these responses are such that not more than 95%, 90%, 80%, 75%, 60%, 40%, or 25%, and most desirably not more than 10% of the cells undergo cell toxicity, cell death, or a decreased ability to carry out a PTGS event, compared to a cell not exposed to such interferon response inhibiting conditions, all other conditions being equal (e.g., same cell type, same transformation with the same dsRNA).

Apoptosis, interferon induction, 2′5′ OAS activation/induction, PKR induction/activation, anti-proliferative responses, and cytopathic effects are all indicators for the RNA stress response pathway. Exemplary assays that can be used to measure the induction of an RNA stress response as described herein include a TUNEL assay to detect apoptotic cells, ELISA assays to detect the induction of alpha, beta and gamma interferon, ribosomal RNA fragmentation analysis to detect activation of 2′5′ OAS, measurement of phosphorylated eIF2a as an indicator of PKR (protein kinase RNA inducible) activation, proliferation assays to detect changes in cellular proliferation, and microscopic analysis of cells to identify cellular cytopathic effects. See, e.g., US Published Application 20040152117, which is herein incorporated by reference in its entirety.

The present invention encompasses methods whereby muscle cells or other competent targeting cells (e.g., respiratory epithelial cells) are transfected with (1) eiRNA or dsRNA or dsRNA complexes and (2) an expression vector encoding a cell-surface ligand that specifically binds to a receptor on a target cell. The eiRNA expression vector and the ligand-encoding expression vector may be a single expression vector or two different expression vectors. Suitable cell surface ligands and target cells include the influenza A hemaglutinin (HA) receptor binding domain which recognizes and interacts with an oligosaccharide on the surface of respiratory epithelial cells. Since avian influenza A viruses and human influenza A viruses preferentially target different epithelial cell-surface oligosaccharide receptors (e.g., epithelial cell receptors identified as glycans terminated by an α2,3-linked sialic acid (SA) that preferentially bind avian strains and glycans terminated by an α2,6-linked SA that bind human strains. J. Virol., August 2006, p. 7469-7480, Vol. 80, No. 15), expression constructs can be designed to express dsRNAs active against human and/or avian influenza A viruses as well as influenza A receptor binding domains that preferentially target the human receptor and/or the avian receptor.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### Identification of Conserved Regions

Influenza viruses are about 80-120 nm in diameter and can be spherical or pleomorphic. They have a lipid membrane envelope that contains the two glycoproteins: hemagglutinin (H) and neuraminidase (N). These two proteins determine the subtypes of Influenza A virus. The Influenza A viral genome consists of eight, single negative-strand RNAs that can range between 890 and 2340 nucleotides long. Each RNA segment encodes one to two proteins.

In GenBank version 150.0 there are more then 16,000 Influenza A sequences from more then 200 different subtypes, and from 12 different hosts (Table 1).

TABLE 1
Distribution of 16015 Influenza A nucleotide sequences

| Influenza A | | No. of | Host | | | | | |
|---|---|---|---|---|---|---|---|---|
| Segment | Length | sequences | Human | Avian | Swine | Equine | Mouse | Other |
| 1 (PB2) | 2341 | 1358 | 631 | 542 | 133 | 33 | 2 | 17 |
| 2 (PB1) | 2341 | 1330 | 647 | 510 | 132 | 27 | 2 | 12 |
| 3 (PA) | 2233 | 1287 | 607 | 522 | 130 | 16 | 2 | 10 |
| 4 (HA) | 1778 | 4495 | 2475 | 1526 | 301 | 100 | 46 | 47 |
| 5 (NP) | 1565 | 1783 | 908 | 629 | 198 | 28 | 4 | 16 |
| 6 (NA) | 1413 | 2172 | 1025 | 913 | 177 | 25 | 6 | 26 |
| 7 (MP) | 1027 | 1847 | 765 | 864 | 153 | 25 | 12 | 28 |
| 8 (NS) | 890 | 1743 | 700 | 821 | 154 | 41 | 9 | 18 |

A segment by segment comparison between all the Influenza A genomes was conducted. Using a modified version of ClustalW, nine multiple alignment schemes were generated:

A. Includes all 1358 Influenza A segment 1 genome sequences

B. Includes all 1330 Influenza A segment 2 genome sequences

C. Includes all 1287 Influenza A segment 3 genome sequences

D. Includes all 4495 Influenza A segment 4 genome sequences

E. Includes all 1783 Influenza A segment 5 genome sequences

F. Includes all 2172 Influenza A segment 6 genome sequences

G. Includes all 1847 Influenza A segment 7 genome sequences

H. Includes all 1743 Influenza A segment 8 genome sequences

I. Includes 1848 Influenza A segment 4 genome sequences

The multiple alignment results were parsed and a table that includes scores for sequence conservation at each position in the Influenza A genome was generated. A sliding window search to identify the longest region of sequence conservation larger then 21 nt in length was created. 14 conserved regions were identified and mapped to GenBank accession numbers: NC_{—}002023.1; NC_{—}002021.1; NC_{—}002022.1; IVI252132; NC_{—}002019.1; CY006189.1; NC_{—}002016.1; NC_{—}002020.1 most of these are the annotated Influenza A reference sequences in RefSeq database.

Complete information regarding the sequence composition of the conserved regions can be found below in Tables 2-15.

The conserved sequences were screened against GenBank sequences (Human, Mouse and Rat cDNA sequence databases).

Segment 2 encoding the polymerase 1, and segment 3 encoding the polymerase PA protein, are the most conserved segments within the influenza A subtypes. Segment 3 has no significant matches to the human, mouse and rat cDNA sequence databases. Segment 4 encoding the hemagglutinin protein and segment 6 encoding the neuraminidase protein were the least conserved segments.

### Influenza A Segment 1

### Segment 1 Conserved Region 1:

(SEQ ID NO: 24)

ATGAC[T(79%)/C(20%)/A(1%)]CC[A(71%)/C(27%)/T(1%)/
G(1%)]AGCAC[A(78%)/G(10%)/C(7%)/T(5%)]GA[G(75%)/
A(24%)/T(1%)]ATGTCA

### Consensus Sequence:

| | |
|---|---|
| ATGACTCCAAGCACAGAGATGTCA | (SEQ ID NO: 25) |

TABLE 2
Base distribution amongst Influenza A segment 1 genomes - Conserved region 1:

| | Location | | | | | | | |
|---|---|---|---|---|---|---|---|---|
| Location on | Within the | | | | | | | |
| NC_002023.1 | alignment | A | C | G | T | Deletions | Total | Consensus |
| 1426 | 856 | 1001 | 0 | 9 | 7 | 264 | 1281 | A |
| 1427 | 857 | 0 | 0 | 8 | 1009 | 264 | 1281 | T |
| 1428 | 858 | 10 | 1 | 1008 | 0 | 262 | 1281 | G |
| 1429 | 859 | 1015 | 1 | 1 | 0 | 264 | 1281 | A |
| 1430 | 860 | 2 | 1015 | 0 | 0 | 264 | 1281 | C |
| 1431 | 861 | 6 | 208 | 1 | 802 | 264 | 1281 | T |
| 1432 | 862 | 0 | 1007 | 0 | 10 | 264 | 1281 | C |
| 1433 | 863 | 0 | 1009 | 0 | 8 | 264 | 1281 | C |
| 1434 | 864 | 730 | 273 | 10 | 13 | 255 | 1281 | A |
| 1435 | 865 | 1017 | 1 | 8 | 0 | 255 | 1281 | A |
| 1436 | 866 | 8 | 0 | 1016 | 1 | 256 | 1281 | G |
| 1437 | 867 | 2 | 946 | 0 | 78 | 255 | 1281 | C |
| 1438 | 868 | 989 | 9 | 25 | 2 | 256 | 1281 | A |
| 1439 | 869 | 5 | 1010 | 0 | 9 | 257 | 1281 | C |
| 1440 | 870 | 794 | 69 | 106 | 48 | 264 | 1281 | A |
| 1441 | 871 | 11 | 1 | 1012 | 0 | 257 | 1281 | G |
| 1442 | 872 | 1010 | 0 | 4 | 2 | 265 | 1281 | A |
| 1443 | 873 | 240 | 1 | 762 | 13 | 265 | 1281 | G |
| 1444 | 874 | 996 | 2 | 16 | 2 | 265 | 1281 | A |
| 1445 | 875 | 4 | 5 | 0 | 1007 | 265 | 1281 | T |
| 1446 | 876 | 8 | 1 | 1006 | 9 | 257 | 1281 | G |
| 1447 | 877 | 2 | 2 | 0 | 1020 | 257 | 1281 | T |
| 1448 | 878 | 0 | 1016 | 1 | 7 | 257 | 1281 | C |
| 1449 | 879 | 959 | 0 | 64 | 1 | 257 | 1281 | A |

Comparing this conserved region to the human cDNA database the following were found: No matches longer than 15 nt; 7 matches of 15 nts; one match of 18 nucleotides with one mismatch.

Comparing Segment 1 Conserved Region 1 to the mouse cDNA database the following were found: no matches longer than 16 nts, two matches of 16 nts, and one match of 18 nts with one mismatch.

Comparing Segment 1 Conserved Region 1 to the rat cDNA database the following were found: no matches longer than 16 nts, 5 matches of 16 nts, and one match of 19 nts with one mismatch.

### Segment 1 Conserved Region 2:

(SEQ ID NO: 26)

GA[G(79%)/A(21%)]GT[C(75%)/T(19%)/G(6%)]AG[T(74%)/
C(26%)]GAAAC[A(88%)/C(11%)G(1%)]CA[G(88%)/A(12%)]
GGAAC[A(75%)/G(15%)/T(10%)]GA[G(84%)/A(16%]A

### Consensus Sequence:

| | |
|---|---|
| GAGGTCAGTGAAACACAGGGAACAGAGA | (SEQ ID NO: 27) |

TABLE 3
Base distribution amongst Influenza A segment 1 genomes - Conserved region 2:

| | Location | | | | | | | |
|---|---|---|---|---|---|---|---|---|
| | Within | | | | | | | |
| Location on | the | | | | | | | |
| NC_002023.1 | alignment | A | C | G | T | Deletions | Total | Consensus |
| 1576 | 1015 | 4 | 0 | 1010 | 0 | 267 | 1281 | G |
| 1577 | 1016 | 1014 | 0 | 0 | 0 | 267 | 1281 | A |
| 1578 | 1017 | 213 | 0 | 801 | 0 | 267 | 1281 | G |
| 1579 | 1018 | 0 | 0 | 994 | 0 | 287 | 1281 | G |
| 1580 | 1019 | 0 | 0 | 0 | 994 | 287 | 1281 | T |
| 1581 | 1020 | 3 | 746 | 54 | 188 | 290 | 1281 | C |
| 1582 | 1021 | 988 | 0 | 0 | 0 | 293 | 1281 | A |
| 1583 | 1022 | 1 | 0 | 987 | 0 | 293 | 1281 | G |
| 1584 | 1023 | 0 | 256 | 0 | 732 | 293 | 1281 | T |
| 1585 | 1024 | 0 | 1 | 995 | 0 | 285 | 1281 | G |
| 1586 | 1025 | 996 | 0 | 0 | 0 | 285 | 1281 | A |
| 1587 | 1026 | 903 | 0 | 93 | 0 | 285 | 1281 | A |
| 1588 | 1027 | 981 | 0 | 15 | 0 | 285 | 1281 | A |
| 1589 | 1028 | 0 | 995 | 0 | 0 | 286 | 1281 | C |
| 1590 | 1029 | 879 | 110 | 6 | 0 | 286 | 1281 | A |
| 1591 | 1030 | 0 | 995 | 0 | 0 | 286 | 1281 | C |
| 1592 | 1031 | 995 | 0 | 0 | 0 | 286 | 1281 | A |
| 1593 | 1032 | 121 | 0 | 875 | 2 | 283 | 1281 | G |
| 1594 | 1033 | 0 | 0 | 998 | 0 | 283 | 1281 | G |
| 1595 | 1034 | 1 | 0 | 997 | 0 | 283 | 1281 | G |
| 1596 | 1035 | 909 | 0 | 87 | 1 | 284 | 1281 | A |
| 1597 | 1036 | 986 | 0 | 3 | 8 | 284 | 1281 | A |
| 1598 | 1037 | 0 | 927 | 0 | 70 | 284 | 1281 | C |
| 1599 | 1038 | 748 | 4 | 153 | 92 | 284 | 1281 | A |
| 1600 | 1039 | 3 | 0 | 990 | 8 | 280 | 1281 | G |
| 1601 | 1040 | 984 | 0 | 11 | 0 | 286 | 1281 | A |
| 1602 | 1041 | 161 | 0 | 834 | 0 | 286 | 1281 | G |
| 1603 | 1042 | 995 | 0 | 0 | 0 | 286 | 1281 | A |

Comparing Conserved Region 2 to the human cDNA database the following were found: no matches of greater than 16 nt; and 3 matches of 16 nt.

Comparing Conserved Region 2 to the mouse cDNA database the following were found: no matches of greater than 15 nt; 3 matches of 16 nt; and three matches with 18 nt including one mismatch.

Comparing Segment 1 Conserved Region 2 to the rat cDNA database the following were found: no matches longer than 17 nts, one match of 17 nts; and three matches of 18 nts with one mismatch.

### Segment 1 Conserved Region 3:

(SEQ ID NO: 28)

GGGCAAGGAGACGT[G(81%)/A(18%)/T(1%)]GTGTTGGTAATGAAA
CG

### Consensus Sequence:

| | |
|---|---|
| GGGCAAGGAGACGTGGTGTTGGTAATGAAACG | (SEQ ID NO: 29) |

TABLE 4
Base distribution amongst Influenza A segment 1 genomes - Conserved region 3:

| | Location | | | | | | | |
|---|---|---|---|---|---|---|---|---|
| | Within | | | | | | | |
| Location on | the | | | | | | | |
| NC_002023.1 | alignment | A | C | G | T | Deletions | Total | Consensus |
| 2206 | 1876 | 9 | 0 | 993 | 0 | 279 | 1281 | G |
| 2207 | 1877 | 0 | 0 | 998 | 2 | 281 | 1281 | G |
| 2208 | 1878 | 39 | 1 | 960 | 0 | 281 | 1281 | G |
| 2209 | 1879 | 9 | 988 | 3 | 0 | 281 | 1281 | C |
| 2210 | 1880 | 990 | 2 | 13 | 0 | 276 | 1281 | A |
| 2211 | 1881 | 998 | 0 | 10 | 4 | 269 | 1281 | A |
| 2212 | 1882 | 15 | 0 | 997 | 0 | 269 | 1281 | G |
| 2213 | 1883 | 2 | 2 | 1008 | 0 | 269 | 1281 | G |
| 2214 | 1884 | 952 | 0 | 60 | 0 | 269 | 1281 | A |
| 2215 | 1885 | 1 | 0 | 1006 | 5 | 269 | 1281 | G |
| 2216 | 1886 | 994 | 8 | 2 | 0 | 277 | 1281 | A |
| 2217 | 1887 | 10 | 969 | 1 | 24 | 277 | 1281 | C |
| 2218 | 1888 | 25 | 3 | 974 | 1 | 277 | 1280 | G |
| 2219 | 1889 | 1 | 10 | 1 | 992 | 277 | 1281 | T |
| 2220 | 1890 | 179 | 0 | 804 | 11 | 287 | 1281 | G |
| 2221 | 1891 | 5 | 0 | 985 | 0 | 291 | 1281 | G |
| 2222 | 1892 | 1 | 1 | 0 | 987 | 292 | 1281 | T |
| 2223 | 1893 | 79 | 8 | 896 | 6 | 292 | 1281 | G |
| 2224 | 1894 | 2 | 11 | 0 | 976 | 292 | 1281 | T |
| 2225 | 1895 | 0 | 9 | 0 | 980 | 292 | 1281 | T |
| 2226 | 1896 | 57 | 1 | 930 | 1 | 292 | 1281 | G |
| 2227 | 1897 | 1 | 0 | 983 | 4 | 293 | 1281 | G |
| 2228 | 1898 | 8 | 1 | 1 | 978 | 293 | 1281 | T |
| 2229 | 1899 | 951 | 0 | 36 | 2 | 292 | 1281 | A |
| 2230 | 1900 | 986 | 0 | 0 | 0 | 295 | 1281 | A |
| 2231 | 1901 | 0 | 5 | 8 | 977 | 291 | 1281 | T |
| 2232 | 1902 | 5 | 2 | 983 | 0 | 291 | 1281 | G |
| 2233 | 1903 | 989 | 0 | 1 | 0 | 291 | 1281 | A |
| 2234 | 1904 | 987 | 0 | 2 | 0 | 292 | 1281 | A |
| 2235 | 1905 | 970 | 8 | 9 | 0 | 294 | 1281 | A |
| 2236 | 1906 | 0 | 985 | 2 | 0 | 294 | 1281 | C |
| 2237 | 1907 | 14 | 0 | 973 | 0 | 294 | 1281 | G |

Comparing Segment 1 Conserved Region 3 to the human cDNA database the following were found: no matches of greater than 15 nucleotides; 4 matches of 15 nucleotides; and 1 match of 19 nucleotides with one mismatch.

Comparing Segment 1 Conserved Region 3 to the mouse cDNA database the following were found: no matches longer than 16 nts, and one match of 16 nts.

Comparing Segment 1 Conserved Region 2 to the rat cDNA database the following were found: no matches longer than 16 nts, three matches of 16 nts, and two matches of 20 nts with one mismatch.

### Influenza A Segment 2

### Segment 2 Conserved Region 4:

(SEQ ID NO: 30)

GACAACATGACCAAGAAAATGGTCACACAAAGAACAATAGG

TABLE 5
Base distribution amongst Influenza A segment 2 genomes - Conserved region 4:

| | Location | | | | | | | |
|---|---|---|---|---|---|---|---|---|
| | Within | | | | | | | |
| Location on | the | | | | | | | |
| NC_002021.1 | alignment | A | C | G | T | Deletions | Total | Consensus |
| 601 | 1056 | 7 | 0 | 1098 | 7 | 141 | 1253 | G |
| 602 | 1057 | 1109 | 1 | 1 | 1 | 141 | 1253 | A |
| 603 | 1058 | 7 | 1100 | 1 | 9 | 136 | 1253 | C |
| 604 | 1059 | 1112 | 3 | 1 | 0 | 137 | 1253 | A |
| 605 | 1060 | 1108 | 0 | 1 | 7 | 137 | 1253 | A |
| 606 | 1061 | 6 | 1041 | 2 | 69 | 135 | 1253 | C |
| 607 | 1062 | 1082 | 1 | 33 | 2 | 135 | 1253 | A |
| 608 | 1063 | 0 | 0 | 0 | 1118 | 135 | 1253 | T |
| 609 | 1064 | 35 | 1 | 1076 | 6 | 135 | 1253 | G |
| 610 | 1065 | 1113 | 1 | 1 | 0 | 137 | 1252 | A |
| 611 | 1066 | 0 | 1106 | 4 | 6 | 137 | 1253 | C |
| 612 | 1067 | 28 | 1039 | 0 | 49 | 137 | 1253 | C |
| 613 | 1068 | 1104 | 4 | 1 | 2 | 142 | 1253 | A |
| 614 | 1069 | 1106 | 0 | 2 | 3 | 142 | 1253 | A |
| 615 | 1070 | 134 | 0 | 967 | 7 | 145 | 1253 | G |
| 616 | 1071 | 1105 | 2 | 1 | 0 | 145 | 1253 | A |
| 617 | 1072 | 1072 | 0 | 33 | 2 | 145 | 1252 | A |
| 618 | 1073 | 1022 | 3 | 74 | 9 | 145 | 1253 | A |
| 619 | 1074 | 1099 | 1 | 7 | 5 | 141 | 1253 | A |
| 620 | 1075 | 7 | 0 | 0 | 1103 | 143 | 1253 | T |
| 621 | 1076 | 1 | 6 | 1103 | 0 | 143 | 1253 | G |
| 622 | 1077 | 16 | 6 | 1087 | 1 | 143 | 1253 | G |
| 623 | 1078 | 5 | 1 | 0 | 1105 | 142 | 1253 | T |
| 624 | 1079 | 44 | 997 | 59 | 10 | 143 | 1253 | C |
| 625 | 1080 | 1097 | 5 | 7 | 1 | 143 | 1253 | A |
| 626 | 1081 | 4 | 1097 | 7 | 4 | 141 | 1253 | C |
| 627 | 1082 | 1054 | 29 | 25 | 2 | 141 | 1251 | A |
| 628 | 1083 | 0 | 1110 | 0 | 2 | 141 | 1253 | C |
| 629 | 1084 | 1101 | 8 | 2 | 1 | 141 | 1253 | A |
| 630 | 1085 | 1009 | 0 | 97 | 6 | 141 | 1253 | A |
| 631 | 1086 | 1070 | 33 | 7 | 2 | 141 | 1253 | A |
| 632 | 1087 | 5 | 9 | 1097 | 0 | 142 | 1253 | G |
| 633 | 1088 | 1025 | 2 | 85 | 0 | 141 | 1253 | A |
| 634 | 1089 | 1098 | 9 | 3 | 1 | 141 | 1252 | A |
| 635 | 1090 | 0 | 1047 | 0 | 2 | 204 | 1253 | C |
| 636 | 1091 | 1014 | 26 | 4 | 6 | 203 | 1253 | A |
| 637 | 1092 | 1030 | 0 | 20 | 0 | 203 | 1253 | A |
| 638 | 1093 | 4 | 10 | 1 | 1035 | 203 | 1253 | T |
| 639 | 1094 | 1031 | 10 | 3 | 6 | 203 | 1253 | A |
| 640 | 1095 | 1 | 7 | 1042 | 0 | 203 | 1253 | G |
| 641 | 1096 | 8 | 1 | 1040 | 0 | 204 | 1253 | G |

Comparing Segment 2 Conserved Region 4 to the human cDNA database the following were found: no matches greater than 17 nt, one match of 17 nt; and two matches of 21 nts with one mismatch.

Comparing Segment 2 Conserved Region 4 to the mouse cDNA database the following were found: no matches greater than 16 nts; 4 matches of 16 nts; one match of 21 nts with one mismatch.

Comparing Segment 2 Conserved Region 4 to the rat cDNA database the following were found: no matches of longer than 19 nts, one match of 19 nts, and one match of 21 nts with one mismatch.

### Influenza A Segment 3

### Segment 3 Conserved Region 5:

(SEQ ID NO: 31)

CG[C(80%)/T(19%)A(1%)]AGGCTTGCCGACCAAAGTCTCCC

### Consensus Sequence:

| | |
|---|---|
| CGCAGGCTTGCCGACCAAAGTCTCCC | (SEQ ID NO: 32) |

TABLE 6
Base distribution amongst Influenza A segment 3 genomes - Conserved region 5:

| | Location | | | | | | | |
|---|---|---|---|---|---|---|---|---|
| | Within | | | | | | | |
| Location on | the | | | | | | | |
| NC_002021.1 | alignment | A | C | G | T | Deletions | Total | Consensus |
| 658 | 1 | 2 | 900 | 0 | 6 | 304 | 1212 | C |
| 659 | 2 | 8 | 1 | 897 | 2 | 304 | 1212 | G |
| 660 | 3 | 9 | 725 | 0 | 174 | 304 | 1212 | C |
| 661 | 4 | 906 | 0 | 0 | 2 | 304 | 1212 | A |
| 662 | 5 | 18 | 1 | 887 | 2 | 304 | 1212 | G |
| 663 | 6 | 76 | 4 | 827 | 1 | 304 | 1212 | G |
| 664 | 7 | 0 | 904 | 3 | 2 | 303 | 1212 | C |
| 665 | 8 | 3 | 0 | 1 | 905 | 303 | 1212 | T |
| 666 | 9 | 4 | 45 | 0 | 858 | 305 | 1212 | T |
| 667 | 10 | 2 | 1 | 904 | 0 | 305 | 1212 | G |
| 668 | 11 | 0 | 904 | 0 | 3 | 305 | 1212 | C |
| 669 | 12 | 42 | 828 | 1 | 36 | 305 | 1212 | C |
| 670 | 13 | 45 | 0 | 862 | 0 | 305 | 1212 | G |
| 671 | 14 | 905 | 0 | 0 | 2 | 305 | 1212 | A |
| 672 | 15 | 3 | 865 | 1 | 37 | 306 | 1212 | C |
| 673 | 16 | 0 | 891 | 0 | 14 | 307 | 1212 | C |
| 674 | 17 | 901 | 0 | 5 | 1 | 305 | 1212 | A |
| 675 | 18 | 891 | 15 | 1 | 0 | 305 | 1212 | A |
| 676 | 19 | 908 | 0 | 0 | 0 | 304 | 1212 | A |
| 677 | 20 | 0 | 0 | 908 | 0 | 304 | 1212 | G |
| 678 | 21 | 0 | 57 | 3 | 854 | 298 | 1212 | T |
| 679 | 22 | 0 | 911 | 2 | 6 | 293 | 1212 | C |
| 680 | 23 | 1 | 0 | 2 | 915 | 294 | 1212 | T |
| 681 | 24 | 4 | 863 | 2 | 50 | 293 | 1212 | C |
| 682 | 25 | 1 | 917 | 1 | 0 | 293 | 1212 | C |
| 683 | 26 | 13 | 904 | 1 | 1 | 293 | 1212 | C |

Comparing Segment 3 Conserved Region 5 to the human cDNA database the following were found: no matches greater than 14 nucleotides; and one match of 14 nts.

Comparing Segment 3 Conserved Region 5 to the mouse cDNA database the following were found: no matches greater than 14 nts; and 3 matches of 14 nts.

Comparing Segment 3 Conserved Region 5 to the rat cDNA database the following were found: no matches greater than 14 nucleotides; 3 matches of 14 nucleotides.

### Segment 3 Conserved Region 6:

| | |
|---|---|
| TTTAGAGCCTATGTGGATGGATT | (SEQ ID NO: 33) |

TABLE 7
Base distribution amongst Influenza A segment 3 genomes - Conserved region 6:

| | Location | | | | | | | |
|---|---|---|---|---|---|---|---|---|
| | Within | | | | | | | |
| Location on | the | | | | | | | |
| NC_002021.1 | alignment | A | C | G | T | Deletions | Total | Consensus |
| 709 | 52 | 2 | 6 | 0 | 906 | 298 | 1212 | T |
| 710 | 53 | 0 | 4 | 6 | 904 | 298 | 1212 | T |
| 711 | 54 | 0 | 6 | 6 | 902 | 298 | 1212 | T |
| 712 | 55 | 906 | 6 | 2 | 0 | 298 | 1212 | A |
| 713 | 56 | 10 | 4 | 900 | 0 | 298 | 1212 | G |
| 714 | 57 | 909 | 1 | 4 | 0 | 298 | 1212 | A |
| 715 | 58 | 17 | 3 | 901 | 0 | 291 | 1212 | G |
| 716 | 59 | 0 | 899 | 0 | 23 | 290 | 1212 | C |
| 717 | 60 | 7 | 898 | 12 | 1 | 294 | 1212 | C |
| 718 | 61 | 0 | 2 | 15 | 902 | 293 | 1212 | T |
| 719 | 62 | 914 | 0 | 5 | 0 | 293 | 1212 | A |
| 720 | 63 | 14 | 0 | 1 | 900 | 297 | 1212 | T |
| 721 | 64 | 3 | 0 | 913 | 0 | 296 | 1212 | G |
| 722 | 65 | 16 | 0 | 0 | 900 | 296 | 1212 | T |
| 723 | 66 | 14 | 13 | 886 | 3 | 296 | 1212 | G |
| 724 | 67 | 0 | 2 | 900 | 14 | 296 | 1212 | G |
| 725 | 68 | 898 | 2 | 0 | 16 | 296 | 1212 | A |
| 726 | 69 | 0 | 0 | 2 | 895 | 315 | 1212 | T |
| 727 | 70 | 2 | 0 | 895 | 0 | 315 | 1212 | G |
| 728 | 71 | 2 | 0 | 893 | 2 | 315 | 1212 | G |
| 729 | 72 | 880 | 2 | 15 | 0 | 315 | 1212 | A |
| 730 | 73 | 0 | 0 | 1 | 896 | 315 | 1212 | T |
| 731 | 74 | 1 | 1 | 2 | 855 | 353 | 1212 | T |

Comparing Segment 3 Conserved Region 6 to the human cDNA database the following were found: no matches of greater than 15 nts, and one match of 15 nucleotides.

Comparing Segment 3 Conserved Region 6 to the mouse cDNA database the following were found: no matches of greater than 14 nts, and 5 matches of 14 nts.

Comparing Segment 3 Conserved Region 6 to the rat cDNA database the following were found: no matches of greater than 18 nts, and one match of 18 nts.

### Influenza A Segment 4

There are 4495 Influenza A genomic sequences for Segment 4, most of which are partial sequences. Aligning all the sequences together resulted in many deletions. Thus, an additional matrix was created containing the 1848 complete segment 4 sequences. Even with this subset segment 4 is highly variable.

### Segment 4 Conserved Region 7:

(SEQ ID NO: 34)

AG[C(57%)/T(32%)/A(11%)]A[A(55%)/C(43%)/T(2%)]TGG
[G(59%)/A(34%)/T(4%)C(3%)]AATCTAATTGCTCC

### Consensus Sequence:

| | |
|---|---|
| AGCAATGGGAATCTAATTGCTCC | (SEQ ID NO: 35) |

TABLE 8
Base distribution amongst Influenza A segment 4 complete genomes - Conserved
region 7:

| | Location | | | | | | | |
|---|---|---|---|---|---|---|---|---|
| | Within | | | | | | | |
| Location on | the | | | | | | | |
| IVI252132 | alignment | A | C | G | T | Deletions | Total | Consensus |
| 816 | 1047 | 996 | 1 | 177 | 308 | 2 | 1484 | A |
| 817 | 1048 | 2 | 380 | 973 | 127 | 2 | 1484 | G |
| 818 | 1049 | 165 | 842 | 0 | 475 | 2 | 1484 | C |
| 819 | 1050 | 1478 | 0 | 6 | 0 | 0 | 1484 | A |
| 820 | 1051 | 816 | 634 | 10 | 24 | 0 | 1484 | A |
| 821 | 1052 | 509 | 59 | 0 | 916 | 0 | 1484 | T |
| 822 | 1053 | 0 | 0 | 1484 | 0 | 0 | 1484 | G |
| 823 | 1054 | 0 | 1 | 1482 | 1 | 0 | 1484 | G |
| 824 | 1055 | 504 | 45 | 876 | 58 | 1 | 1484 | G |
| 825 | 1056 | 1336 | 0 | 146 | 1 | 1 | 1484 | A |
| 826 | 1057 | 1337 | 130 | 16 | 0 | 1 | 1484 | A |
| 827 | 1058 | 38 | 251 | 8 | 1187 | 0 | 1484 | T |
| 828 | 1059 | 0 | 952 | 1 | 531 | 0 | 1484 | C |
| 829 | 1060 | 0 | 0 | 1 | 1483 | 0 | 1484 | T |
| 830 | 1061 | 1019 | 329 | 29 | 107 | 0 | 1484 | A |
| 831 | 1062 | 1371 | 4 | 96 | 13 | 0 | 1484 | A |
| 832 | 1063 | 0 | 1 | 0 | 1483 | 0 | 1484 | T |
| 832 | 1064 | 325 | 141 | 60 | 958 | 0 | 1484 | T |
| 834 | 1065 | 0 | 0 | 1484 | 0 | 0 | 1484 | G |
| 835 | 1066 | 0 | 1440 | 0 | 44 | 0 | 1484 | C |
| 836 | 1067 | 185 | 96 | 103 | 1100 | 0 | 1484 | T |
| 837 | 1068 | 1 | 1480 | 0 | 3 | 0 | 1484 | C |
| 838 | 1069 | 0 | 1482 | 0 | 1 | 1 | 1484 | C |

Comparing Segment 4 Conserved Region 7 to the human cDNA database the following were found: no matches of longer than 15 nts, and two matches of 15 nts.

Comparing Segment 4 Conserved Region 7 to the mouse cDNA database the following were found: no matches of longer than 14 nts, and two matches of 14 nts.

Comparing Segment 4 Conserved Region 7 to the rat cDNA database the following were found: no matches longer than 13 nts, and seven matches of 13 nts.

### Influenza A Segment 5

### Segment 5 Conserved Region 8:

(SEQ ID NO: 36)

ATGGCGTC[T(59%)/C(41%)]CAAGGCACCAAACG[A(57%)/G
(43%)]TCTTATGA[A(79%)/G(21%]CA

### Consensus Sequence:

(SEQ ID NO: 37)

ATGGCGTCTCAAGGCACCAAACGATCTTATGAACA

TABLE 9
Base distribution amongst Influenza A segment 5 genomes - Conserved region 8:

| | Location | | | | | | | |
|---|---|---|---|---|---|---|---|---|
| | Within | | | | | | | |
| Location on | the | | | | | | | |
| NC_002019.1 | alignment | A | C | G | T | Deletions | Total | Consensus |
| 46 | 61 | 1323 | 1 | 2 | 0 | 377 | 1703 | A |
| 47 | 62 | 0 | 2 | 0 | 1321 | 380 | 1703 | T |
| 48 | 63 | 0 | 0 | 1326 | 0 | 377 | 1703 | G |
| 49 | 64 | 1 | 7 | 1316 | 3 | 376 | 1703 | G |
| 50 | 65 | 0 | 1335 | 0 | 2 | 366 | 1703 | C |
| 51 | 66 | 23 | 4 | 1306 | 4 | 366 | 1703 | G |
| 52 | 67 | 15 | 87 | 2 | 1234 | 365 | 1703 | T |
| 53 | 68 | 5 | 1249 | 1 | 85 | 363 | 1703 | C |
| 54 | 69 | 3 | 554 | 1 | 782 | 363 | 1703 | t |
| 55 | 70 | 2 | 1320 | 2 | 17 | 362 | 1703 | C |
| 56 | 71 | 1325 | 2 | 18 | 0 | 358 | 1703 | A |
| 57 | 72 | 1319 | 18 | 6 | 1 | 359 | 1703 | A |
| 58 | 73 | 4 | 0 | 1329 | 16 | 354 | 1703 | G |
| 59 | 74 | 0 | 3 | 1348 | 0 | 352 | 1703 | G |
| 60 | 75 | 0 | 1349 | 0 | 3 | 351 | 1703 | C |
| 61 | 76 | 1349 | 1 | 2 | 0 | 351 | 1703 | A |
| 62 | 77 | 10 | 1324 | 3 | 16 | 350 | 1703 | C |
| 63 | 78 | 4 | 1328 | 0 | 22 | 349 | 1703 | C |
| 64 | 79 | 1331 | 2 | 6 | 17 | 347 | 1703 | A |
| 65 | 80 | 1333 | 4 | 20 | 0 | 346 | 1703 | A |
| 66 | 81 | 1348 | 1 | 2 | 5 | 347 | 1703 | A |
| 67 | 82 | 16 | 1365 | 8 | 1 | 313 | 1703 | C |
| 68 | 83 | 2 | 2 | 1387 | 0 | 312 | 1703 | G |
| 69 | 84 | 792 | 0 | 601 | 0 | 310 | 1703 | a |
| 70 | 85 | 0 | 20 | 0 | 1373 | 310 | 1703 | T |
| 71 | 86 | 0 | 1378 | 0 | 17 | 308 | 1703 | C |
| 72 | 87 | 87 | 62 | 0 | 1246 | 308 | 1703 | T |
| 73 | 88 | 7 | 1 | 0 | 1372 | 323 | 1703 | T |
| 74 | 89 | 1375 | 0 | 8 | 0 | 320 | 1703 | A |
| 75 | 90 | 1 | 97 | 1 | 1284 | 320 | 1703 | T |
| 76 | 91 | 1 | 1 | 1381 | 0 | 320 | 1703 | G |
| 77 | 92 | 1381 | 2 | 2 | 0 | 318 | 1703 | A |
| 78 | 93 | 1093 | 0 | 292 | 0 | 318 | 1703 | A |
| 79 | 94 | 2 | 1376 | 0 | 1 | 324 | 1703 | C |
| 80 | 95 | 1379 | 0 | 1 | 0 | 323 | 1703 | A |

Comparing Segment 5 Conserved Region 8 to the human cDNA database the following were found: no matches of longer than 16 nts, and one match of 16 nts.

Comparing Segment 5 Conserved Region 8 to the mouse cDNA database the following were found: no matches greater than 14 nucleotides, four matches of 14 nts, and one match of 18 nts with one mismatch.

Comparing Segment 5 Conserved Region 8 to the rat cDNA database the following were found: no matches greater than 14 nts long, and two matches of 14 nts.

### Segment 5 Conserved Region 9:

(SEQ ID NO: 38)

TCA[G(61%)/A(39%)][C(71%)/T(29%)]TGGT[G(77%)/A
(23%)]TGGATGGCATGCCATT

### Consensus Sequence:

| | |
|---|---|
| TCAGCTGGTGTGGATGGCATGCCATT | (SEQ ID NO: 39) |

TABLE 10
Base distribution amongst Influenza A segment 5 genomes - Conserved region 9:

| | Location | | | | | | | |
|---|---|---|---|---|---|---|---|---|
| | Within | | | | | | | |
| Location on | the | | | | | | | |
| NC_002019.1 | alignment | A | C | G | T | Deletions | Total | Consensus |
| 1023 | 1848 | 0 | 97 | 4 | 1320 | 282 | 1703 | T |
| 1024 | 1849 | 0 | 1403 | 0 | 11 | 289 | 1703 | C |
| 1025 | 1850 | 1394 | 0 | 0 | 0 | 309 | 1703 | A |
| 1026 | 1851 | 534 | 1 | 851 | 1 | 316 | 1703 | g |
| 1027 | 1852 | 1 | 978 | 0 | 406 | 318 | 1703 | c |
| 1028 | 1853 | 1 | 0 | 4 | 1380 | 318 | 1703 | T |
| 1029 | 1854 | 135 | 0 | 1249 | 0 | 319 | 1703 | G |
| 1030 | 1855 | 42 | 0 | 1339 | 0 | 322 | 1703 | G |
| 1031 | 1856 | 2 | 5 | 0 | 1371 | 325 | 1703 | T |
| 1032 | 1857 | 312 | 5 | 1057 | 4 | 325 | 1703 | G |
| 1033 | 1858 | 2 | 4 | 0 | 1371 | 326 | 1703 | T |
| 1034 | 1859 | 0 | 0 | 1376 | 1 | 326 | 1703 | G |
| 1035 | 1860 | 3 | 0 | 1369 | 0 | 331 | 1703 | G |
| 1036 | 1861 | 1369 | 2 | 2 | 0 | 330 | 1703 | A |
| 1037 | 1862 | 0 | 1 | 0 | 1372 | 330 | 1703 | T |
| 1038 | 1863 | 0 | 0 | 1374 | 0 | 329 | 1703 | G |
| 1039 | 1864 | 1 | 0 | 1364 | 1 | 337 | 1703 | G |
| 1040 | 1865 | 0 | 1365 | 0 | 1 | 337 | 1703 | C |
| 1041 | 1866 | 1346 | 1 | 6 | 12 | 338 | 1703 | A |
| 1042 | 1867 | 1 | 1 | 0 | 1360 | 341 | 1703 | T |
| 1043 | 1868 | 0 | 4 | 1356 | 0 | 343 | 1703 | G |
| 1044 | 1869 | 1 | 1254 | 0 | 105 | 343 | 1703 | C |
| 1045 | 1870 | 146 | 1208 | 1 | 1 | 347 | 1703 | C |
| 1046 | 1871 | 1353 | 0 | 0 | 1 | 349 | 1703 | A |
| 1047 | 1872 | 1 | 236 | 0 | 1116 | 350 | 1703 | T |
| 1048 | 1873 | 0 | 1 | 0 | 1351 | 351 | 1703 | T |

Comparing Segment 5 Conserved Region 9 to the human cDNA database the following were found: no matches longer than 16 nts, and one of 16 nts.

Comparing Segment 5 Conserved Region 9 to the mouse cDNA database the following were found: no matches greater than 15 nt long, three matches of 15 nts, and one match of 18 nts with one mismatch.

Comparing Segment 5 Conserved Region 9 to the rat cDNA database the following were found: no matches of longer than 15 nts, three matches of 15 nts, one match of 18 nts with one mismatch.

### Influenza A Segment 6

### Segment 6 Conserved Region 10:

(SEQ ID NO: 40)

TCCAG[T(63%)/A(32%)/G(4%)C(1%)]TA[T(88%)/C(12%)]
[G(65%)/A(30%)/T(5%)]T[G(64%)/A(30%)/C(5%)/T(1%)]
TGC[T(63%)/A(34%)/G(3%)]C[A(64%)/T(31%)/C(3%)/G
(2%)]GGA

### Consensus Sequence:

| | |
|---|---|
| TCCAGTTATGTGTGCTCAGGA | (SEQ ID NO: 41) |

TABLE 11
Base distribution amongst Influenza A segment 6 genomes - Conserved region 10:

| | Location | | | | | | | |
|---|---|---|---|---|---|---|---|---|
| | Within | | | | | | | |
| Location on | the | | | | | | | |
| CY006189.1 | alignment | A | C | G | T | Deletions | Total | Consensus |
| 937 | 1277 | 68 | 0 | 22 | 1166 | 830 | 2086 | T |
| 938 | 1278 | 0 | 1227 | 6 | 23 | 830 | 2086 | C |
| 939 | 1279 | 54 | 1178 | 3 | 46 | 805 | 2086 | C |
| 940 | 1280 | 1181 | 15 | 125 | 0 | 765 | 2086 | A |
| 941 | 1281 | 28 | 4 | 1854 | 0 | 200 | 2086 | G |
| 942 | 1282 | 606 | 14 | 82 | 1178 | 206 | 2086 | T |
| 943 | 1283 | 3 | 17 | 0 | 1871 | 195 | 2086 | T |
| 944 | 1284 | 1888 | 0 | 3 | 0 | 195 | 2086 | A |
| 945 | 1285 | 2 | 228 | 0 | 1662 | 194 | 2086 | T |
| 946 | 1286 | 557 | 1 | 1234 | 100 | 194 | 2086 | G |
| 947 | 1287 | 5 | 0 | 1 | 1886 | 194 | 2086 | T |
| 948 | 1288 | 565 | 99 | 1217 | 10 | 195 | 2086 | G |
| 949 | 1289 | 0 | 2 | 17 | 1872 | 195 | 2086 | T |
| 950 | 1290 | 5 | 0 | 1885 | 0 | 196 | 2086 | G |
| 951 | 1291 | 1 | 1731 | 0 | 156 | 198 | 2086 | C |
| 952 | 1292 | 635 | 1 | 56 | 1196 | 198 | 2086 | T |
| 953 | 1293 | 5 | 1260 | 617 | 2 | 202 | 2086 | C |
| 954 | 1294 | 1212 | 63 | 36 | 573 | 202 | 2086 | A |
| 955 | 1295 | 82 | 9 | 1789 | 1 | 205 | 2086 | G |
| 956 | 1296 | 101 | 6 | 1769 | 0 | 210 | 2086 | G |
| 957 | 1297 | 1348 | 27 | 475 | 27 | 209 | 2086 | A |

Comparing Segment 6 Conserved Region 10 to the human cDNA database the following were found: no matches of greater than 16 nts, two matches of 16 nts, and one match of 18 nts with one mismatch.

Comparing Segment 6 Conserved Region 10 to the mouse cDNA database the following were found: no match greater than 14 nts, and seven matches of 14 nts.

Comparing Segment 6 Conserved Region 10 to the rat cDNA database the following were found: no matches longer than 14 nts, three matches of 14 nts, and one match of 18 nts with one mismatch.

### Influenza A Segment 7

### Segment 7 Conserved Region 11:

(SEQ ID NO: 42)

AGGCCCCCTCAAAGCCGA[G(73%)/A(27%)]ATCGC[G(76%)/T
(13%)/A(11%)]CAGA[G(78%)/C(13%)/A(9%)]ACTTGAA

### Consensus Sequence:

(SEQ ID NO: 43)

AGGCCCCCTCAAAGCCGAGATCGCGCAGAGACTTGAA

TABLE 12
Base distribution amongst Influenza A segment 7 genomes - Conserved region 11:

| | Location | | | | | | | |
|---|---|---|---|---|---|---|---|---|
| | Within | | | | | | | |
| Location on | the | | | | | | | |
| NC_002016.1 | alignment | A | C | G | T | Deletions | Total | Consensus |
| 76 | 222 | 1381 | 2 | 15 | 0 | 372 | 1770 | A |
| 77 | 223 | 0 | 0 | 1396 | 0 | 372 | 1768 | G |
| 78 | 224 | 0 | 0 | 1408 | 0 | 362 | 1770 | G |
| 79 | 225 | 129 | 1406 | 0 | 1 | 234 | 1770 | C |
| 80 | 226 | 136 | 1411 | 0 | 0 | 223 | 1770 | C |
| 81 | 227 | 1 | 1543 | 2 | 3 | 221 | 1770 | C |
| 82 | 228 | 5 | 1412 | 135 | 1 | 217 | 1770 | C |
| 83 | 229 | 0 | 1411 | 1 | 1 | 357 | 1770 | C |
| 84 | 230 | 2 | 1 | 0 | 1410 | 357 | 1770 | T |
| 85 | 231 | 0 | 1413 | 1 | 0 | 356 | 1770 | C |
| 86 | 232 | 1415 | 0 | 1 | 0 | 354 | 1770 | A |
| 87 | 233 | 1414 | 1 | 1 | 0 | 354 | 1770 | A |
| 88 | 234 | 1413 | 1 | 0 | 1 | 355 | 1770 | A |
| 89 | 235 | 4 | 0 | 1412 | 0 | 354 | 1770 | G |
| 90 | 236 | 1 | 1413 | 1 | 1 | 354 | 1770 | C |
| 91 | 237 | 1 | 1414 | 1 | 0 | 354 | 1770 | C |
| 92 | 238 | 1 | 7 | 1408 | 0 | 354 | 1770 | G |
| 93 | 239 | 1416 | 0 | 0 | 0 | 354 | 1770 | A |
| 94 | 240 | 386 | 1 | 1031 | 0 | 352 | 1770 | G |
| 95 | 241 | 1352 | 1 | 2 | 206 | 209 | 1770 | A |
| 96 | 242 | 199 | 0 | 10 | 1354 | 207 | 1770 | T |
| 97 | 243 | 73 | 1430 | 0 | 60 | 207 | 1770 | C |
| 98 | 244 | 1 | 2 | 1557 | 3 | 207 | 1770 | G |
| 99 | 245 | 0 | 1352 | 1 | 210 | 207 | 1770 | C |
| 100 | 246 | 176 | 5 | 1178 | 205 | 206 | 1770 | G |
| 101 | 247 | 0 | 1556 | 0 | 10 | 204 | 1770 | C |
| 102 | 248 | 1355 | 0 | 3 | 209 | 203 | 1770 | A |
| 103 | 249 | 1 | 199 | 1356 | 9 | 205 | 1770 | G |
| 104 | 250 | 1354 | 1 | 4 | 206 | 205 | 1770 | A |
| 105 | 251 | 139 | 199 | 1213 | 9 | 210 | 1770 | G |
| 106 | 252 | 1342 | 1 | 13 | 206 | 208 | 1770 | A |
| 107 | 253 | 208 | 1353 | 0 | 2 | 207 | 1770 | C |
| 108 | 254 | 2 | 2 | 0 | 1559 | 207 | 1770 | T |
| 109 | 255 | 3 | 234 | 19 | 1307 | 207 | 1770 | T |
| 110 | 256 | 2 | 1 | 1358 | 0 | 409 | 1770 | G |
| 111 | 257 | 1361 | 0 | 0 | 1 | 408 | 1770 | A |
| 112 | 258 | 1266 | 0 | 93 | 3 | 408 | 1770 | A |

Comparing Segment 7 Conserved Region 11 to the human cDNA database the following were found: no matches longer than 16 nts, and three matches of 16 nts.

Comparing Segment 7 Conserved Region 11 to the mouse cDNA database the following were found: no matches longer than 15 nts, and one match of 15 nts.

Comparing Segment 7 Conserved Region 11 to the rat cDNA database the following were found: no matches greater than 15 nts, and one match of 15 nts.

### Segment 7 Conserved Region 12:

(SEQ ID NO: 44)

TTTGT[G(81%)/A(19%)]TTCACGCTCACCGTGCCCAGTGAGCG
[A(83%)/G(17%)]

### Consensus Sequence:

| | |
|---|---|
| TTTGTGTTCACGCTCACCGTGCCCAGTGAGCGA | (SEQ ID NO: 45) |

TABLE 13
Base distribution amongst Influenza A segment 7 genomes - Conserved region 12:

| | Location | | | | | | | |
|---|---|---|---|---|---|---|---|---|
| | Within | | | | | | | |
| Location on | the | | | | | | | |
| NC_002016.1 | alignment | A | C | G | T | Deletions | Total | Consensus |
| 209 | 458 | 1 | 2 | 0 | 1200 | 567 | 1770 | T |
| 210 | 459 | 0 | 0 | 1 | 1202 | 567 | 1770 | T |
| 211 | 460 | 0 | 38 | 1 | 1166 | 565 | 1770 | T |
| 212 | 461 | 1 | 1 | 1201 | 1 | 565 | 1769 | G |
| 213 | 462 | 3 | 0 | 0 | 1202 | 565 | 1770 | T |
| 214 | 463 | 226 | 2 | 976 | 4 | 562 | 1770 | G |
| 215 | 464 | 1 | 1 | 1 | 1206 | 561 | 1770 | T |
| 216 | 465 | 434 | 4 | 1 | 1205 | 126 | 1770 | T |
| 217 | 466 | 0 | 1564 | 0 | 80 | 126 | 1770 | C |
| 218 | 467 | 1207 | 14 | 4 | 420 | 125 | 1770 | A |
| 219 | 468 | 432 | 1205 | 4 | 1 | 128 | 1770 | C |
| 220 | 469 | 3 | 0 | 1200 | 0 | 567 | 1770 | G |
| 221 | 470 | 0 | 1203 | 0 | 0 | 567 | 1770 | C |
| 222 | 471 | 0 | 0 | 0 | 1203 | 567 | 1770 | T |
| 223 | 472 | 1 | 1201 | 1 | 0 | 567 | 1770 | C |
| 224 | 473 | 1200 | 1 | 1 | 1 | 567 | 1770 | A |
| 225 | 474 | 1 | 1202 | 0 | 0 | 567 | 1770 | C |
| 226 | 475 | 0 | 1203 | 0 | 0 | 567 | 1770 | C |
| 227 | 476 | 1 | 0 | 1202 | 0 | 567 | 1770 | G |
| 228 | 477 | 1 | 0 | 0 | 1202 | 567 | 1770 | T |
| 229 | 478 | 1 | 0 | 1202 | 0 | 567 | 1770 | G |
| 230 | 479 | 0 | 1202 | 0 | 1 | 567 | 1770 | C |
| 231 | 480 | 0 | 1202 | 1 | 0 | 567 | 1770 | C |
| 232 | 481 | 0 | 1132 | 1 | 1 | 636 | 1770 | C |
| 233 | 482 | 1131 | 0 | 2 | 1 | 636 | 1770 | A |
| 234 | 483 | 0 | 2 | 1132 | 0 | 636 | 1770 | G |
| 235 | 484 | 0 | 14 | 0 | 1121 | 635 | 1770 | T |
| 236 | 485 | 1 | 2 | 1133 | 0 | 634 | 1770 | G |
| 237 | 486 | 1135 | 0 | 1 | 0 | 634 | 1770 | A |
| 238 | 487 | 31 | 0 | 1105 | 0 | 634 | 1770 | G |
| 239 | 488 | 0 | 1134 | 0 | 0 | 636 | 1770 | C |
| 240 | 489 | 6 | 0 | 1127 | 0 | 636 | 1769 | G |
| 241 | 490 | 941 | 0 | 192 | 0 | 636 | 1769 | A |

Comparing Segment 7 Conserved Region 12 to the human cDNA database the following were found: no matches of longer than 15 nts, and four matches of 15 nts.

Comparing Segment 7 Conserved Region 12 to the mouse cDNA database the following were found: no matches of longer than 15 nts, and one match of 15 nts

Comparing Segment 7 Conserved Region 12 to the rat cDNA database the following were found: no matches of greater than 15 nts, and one match of 15 nts.

### Segment 7 Conserved Region 13:

(SEQ ID NO: 46)

AGATGATCTTCTTGAAAATTTGCAG[G(66%)/A(27%)/C(7%)]CCTA
[C(60%)/T(4%)]CAGAAACG[A(59%)/G(41%)]ATGGG

### Consensus Sequence:

(SEQ ID NO: 47)

AGATGATCTTCTTGAAAATTTGCAGGCCTACCAGAAACGAATGGG

TABLE 14
Base distribution amongst Influenza A segment 7 genomes - Conserved region 13:

| | Location | | | | | | | |
|---|---|---|---|---|---|---|---|---|
| Location on | Within the | | | | | | | |
| NC_002016.1 | alignment | A | C | G | T | Deletions | Total | Consensus |
| 715 | 167 | 1600 | 2 | 21 | 4 | 143 | 1770 | A |
| 716 | 168 | 55 | 3 | 1527 | 45 | 140 | 1770 | G |
| 717 | 169 | 1574 | 5 | 48 | 3 | 140 | 1770 | A |
| 718 | 170 | 48 | 4 | 0 | 1579 | 139 | 1770 | T |
| 719 | 171 | 242 | 1 | 1334 | 1 | 192 | 1770 | G |
| 720 | 172 | 1575 | 0 | 3 | 0 | 192 | 1770 | A |
| 721 | 173 | 0 | 57 | 2 | 1519 | 192 | 1770 | T |
| 722 | 174 | 0 | 1574 | 1 | 1 | 193 | 1769 | C |
| 723 | 175 | 3 | 1 | 1 | 1574 | 191 | 1770 | T |
| 724 | 176 | 2 | 84 | 0 | 1493 | 191 | 1770 | T |
| 725 | 177 | 170 | 1401 | 2 | 5 | 192 | 1770 | C |
| 726 | 178 | 0 | 0 | 0 | 1578 | 192 | 1770 | T |
| 727 | 179 | 0 | 4 | 0 | 1573 | 193 | 1770 | T |
| 728 | 180 | 1 | 4 | 1616 | 0 | 149 | 1770 | G |
| 729 | 181 | 1562 | 3 | 59 | 1 | 145 | 1770 | A |
| 730 | 182 | 1591 | 3 | 28 | 3 | 145 | 1770 | A |
| 731 | 183 | 1510 | 113 | 0 | 3 | 144 | 1770 | A |
| 732 | 184 | 1542 | 78 | 4 | 1 | 145 | 1770 | A |
| 733 | 185 | 96 | 11 | 15 | 1502 | 146 | 1770 | T |
| 734 | 186 | 5 | 10 | 0 | 1604 | 151 | 1770 | T |
| 735 | 187 | 5 | 1 | 1 | 1616 | 147 | 1770 | T |
| 736 | 188 | 75 | 3 | 1545 | 0 | 147 | 1770 | G |
| 737 | 189 | 4 | 1509 | 107 | 3 | 147 | 1770 | C |
| 738 | 190 | 1534 | 0 | 91 | 0 | 145 | 1770 | A |
| 739 | 191 | 111 | 3 | 1511 | 0 | 145 | 1770 | G |
| 740 | 192 | 446 | 107 | 1064 | 7 | 146 | 1770 | G |
| 741 | 193 | 6 | 1513 | 0 | 102 | 149 | 1770 | C |
| 742 | 194 | 75 | 1455 | 4 | 83 | 153 | 1770 | C |
| 743 | 195 | 105 | 5 | 0 | 1506 | 154 | 1770 | T |
| 744 | 196 | 1520 | 13 | 0 | 83 | 154 | 1770 | A |
| 745 | 197 | 0 | 963 | 0 | 652 | 155 | 1770 | C |
| 746 | 198 | 0 | 1611 | 0 | 4 | 155 | 1770 | C |
| 747 | 199 | 1506 | 4 | 0 | 105 | 155 | 1770 | A |
| 748 | 200 | 148 | 1 | 1466 | 0 | 154 | 1769 | G |
| 749 | 201 | 1510 | 0 | 106 | 0 | 154 | 1770 | A |
| 750 | 202 | 1501 | 87 | 7 | 22 | 153 | 1770 | A |
| 751 | 203 | 1453 | 12 | 51 | 101 | 153 | 1770 | A |
| 752 | 204 | 16 | 1600 | 0 | 1 | 153 | 1770 | C |
| 753 | 205 | 0 | 101 | 1506 | 10 | 153 | 1770 | G |
| 754 | 206 | 956 | 0 | 657 | 5 | 152 | 1770 | A |
| 755 | 207 | 1506 | 5 | 106 | 0 | 153 | 1770 | A |
| 756 | 208 | 0 | 8 | 1 | 1608 | 153 | 1770 | T |
| 757 | 209 | 20 | 0 | 1597 | 1 | 152 | 1770 | G |
| 758 | 210 | 0 | 110 | 1506 | 1 | 153 | 1770 | G |
| 759 | 211 | 0 | 0 | 1506 | 0 | 264 | 1770 | G |

Comparing Segment 7 Conserved Region 13 to the human cDNA database the following were found: no matches greater than 16 nts, one match of 16 nts, and two matches of 18 nts with one mismatch.

Comparing Segment 7 Conserved Region 13 to the mouse cDNA database the following were found: no matches longer than 15 nts, and four matches of 15 nts.

Comparing Segment 7 Conserved Region 13 to the rat cDNA database the following were found: no matches of longer than 15 nts, and four matches of 15 nts.

### Influenza A Segment 8

### Segment 8 Conserved Region 14:

(SEQ ID NO: 48)

GAGGATGTCAAAAATGCAATTGGGGTCCTCATC[G(73%)/A(16%)/
C(11%)][G(73%)/T(25%)/A(2%)]AGG[A(59%)/G(41%)]
[C(73%)/A(24%)/T(3%)]TTGAATGGA

### Consensus Sequence:

(SEQ ID NO: 49)

GAGGATGTCAAAAATGCAATTGGGGTCCTCATCGGAGGACTTGAATGGA

TABLE 15
Base distribution amongst Influenza A segment 8 genomes - Conserved region 14:

| | Location | | | | | | | |
|---|---|---|---|---|---|---|---|---|
| | Within | | | | | | | |
| Location on | the | | | | | | | |
| NC_002020.1 | alignment | A | C | G | T | Deletions | Total | Consensus |
| 540 | 732 | 221 | 0 | 1398 | 0 | 49 | 1668 | G |
| 541 | 733 | 1213 | 0 | 2 | 404 | 49 | 1668 | A |
| 542 | 734 | 0 | 1 | 1215 | 403 | 49 | 1668 | G |
| 543 | 735 | 11 | 396 | 1212 | 0 | 49 | 1668 | G |
| 544 | 736 | 1220 | 0 | 399 | 0 | 49 | 1668 | A |
| 545 | 737 | 404 | 0 | 0 | 1215 | 49 | 1668 | T |
| 546 | 738 | 8 | 1 | 1610 | 0 | 49 | 1668 | G |
| 547 | 739 | 0 | 221 | 0 | 1398 | 49 | 1668 | T |
| 548 | 740 | 0 | 1396 | 223 | 0 | 49 | 1668 | C |
| 549 | 741 | 1216 | 1 | 0 | 402 | 49 | 1668 | A |
| 550 | 742 | 1216 | 403 | 0 | 0 | 49 | 1668 | A |
| 551 | 743 | 1213 | 0 | 2 | 404 | 49 | 1668 | A |
| 552 | 744 | 1216 | 1 | 402 | 0 | 49 | 1668 | A |
| 553 | 745 | 1609 | 1 | 0 | 9 | 49 | 1668 | A |
| 554 | 746 | 0 | 0 | 0 | 1215 | 453 | 1668 | T |
| 555 | 747 | 0 | 0 | 1215 | 0 | 453 | 1668 | G |
| 556 | 748 | 0 | 1215 | 0 | 0 | 453 | 1668 | C |
| 557 | 749 | 1207 | 0 | 8 | 0 | 453 | 1668 | A |
| 558 | 750 | 1129 | 0 | 86 | 0 | 453 | 1668 | A |
| 559 | 751 | 0 | 0 | 0 | 1215 | 453 | 1668 | T |
| 560 | 752 | 0 | 0 | 0 | 1215 | 453 | 1668 | T |
| 561 | 753 | 2 | 1 | 1212 | 0 | 453 | 1668 | G |
| 562 | 754 | 10 | 0 | 1205 | 0 | 453 | 1668 | G |
| 563 | 755 | 19 | 163 | 1033 | 0 | 453 | 1668 | G |
| 564 | 756 | 33 | 1 | 1181 | 0 | 453 | 1668 | G |
| 565 | 757 | 2 | 8 | 1 | 1204 | 453 | 1668 | T |
| 566 | 758 | 1 | 1213 | 0 | 1 | 453 | 1668 | C |
| 567 | 759 | 0 | 1215 | 0 | 0 | 453 | 1668 | C |
| 568 | 760 | 0 | 1 | 0 | 1214 | 453 | 1668 | T |
| 569 | 761 | 401 | 1214 | 4 | 0 | 49 | 1668 | C |
| 570 | 762 | 1590 | 0 | 29 | 0 | 49 | 1668 | A |
| 571 | 763 | 234 | 161 | 3 | 1221 | 49 | 1668 | T |
| 572 | 764 | 0 | 1216 | 0 | 403 | 49 | 1668 | C |
| 573 | 765 | 256 | 179 | 1183 | 1 | 49 | 1668 | G |
| 574 | 766 | 31 | 0 | 1185 | 403 | 49 | 1668 | G |
| 575 | 767 | 1547 | 0 | 69 | 3 | 49 | 1668 | A |
| 576 | 768 | 0 | 404 | 1214 | 0 | 49 | 1667 | G |
| 577 | 769 | 376 | 0 | 1214 | 0 | 77 | 1667 | G |
| 578 | 770 | 944 | 1 | 645 | 0 | 77 | 1667 | A |
| 579 | 771 | 376 | 1162 | 0 | 51 | 77 | 1666 | C |
| 580 | 772 | 0 | 2 | 0 | 1213 | 451 | 1666 | T |
| 581 | 773 | 0 | 0 | 0 | 1217 | 451 | 1668 | T |
| 582 | 774 | 38 | 0 | 1179 | 0 | 451 | 1668 | G |
| 583 | 775 | 1217 | 0 | 0 | 0 | 451 | 1668 | A |
| 584 | 776 | 1204 | 0 | 13 | 0 | 451 | 1668 | A |
| 585 | 777 | 0 | 0 | 0 | 1217 | 451 | 1668 | T |
| 586 | 778 | 6 | 0 | 1587 | 0 | 75 | 1668 | G |
| 587 | 779 | 376 | 0 | 1217 | 0 | 75 | 1668 | G |
| 588 | 780 | 1216 | 1 | 0 | 376 | 75 | 1668 | A |

Comparing Segment 8 Conserved Region 14 to the human cDNA database the following were found: no matches of longer than 16 nts, and two matches of 16 nts.

Comparing Segment 8 Conserved Region 14 to the mouse cDNA database the following were found: no matches greater than 16 nts, two matches of 16 nts, and one match of 20 nts with one mismatch.

Comparing Segment 8 Conserved Region 14 to the rat cDNA database the following were found: no matches of longer than 15 nts, two matches of 15 nts, and one match of 21 nts with one mismatch.

Interestingly, Segment 4 encoding the hemagglutinin protein, and Segment 6 encoding the neuraminidase protein, were the least conserved segments. These proteins, which determine the Influenza A subtype and are the targets for host immune surveillance, are generally under positive selection pressure.

### Example 2

### Exemplary Influenza siRNAs

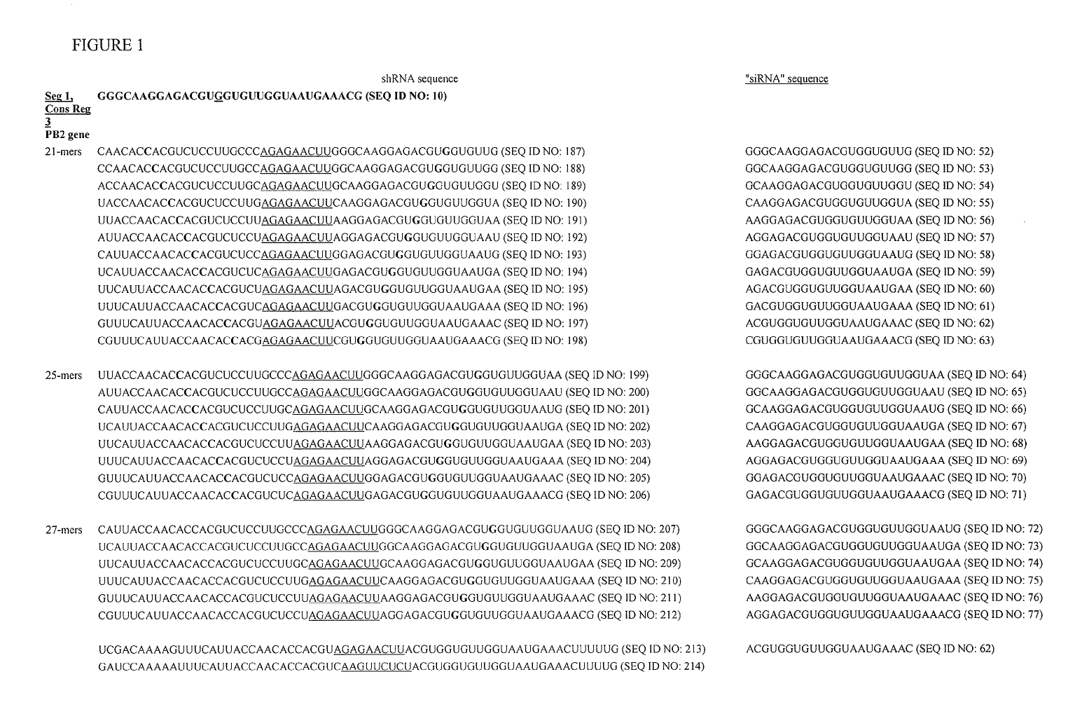

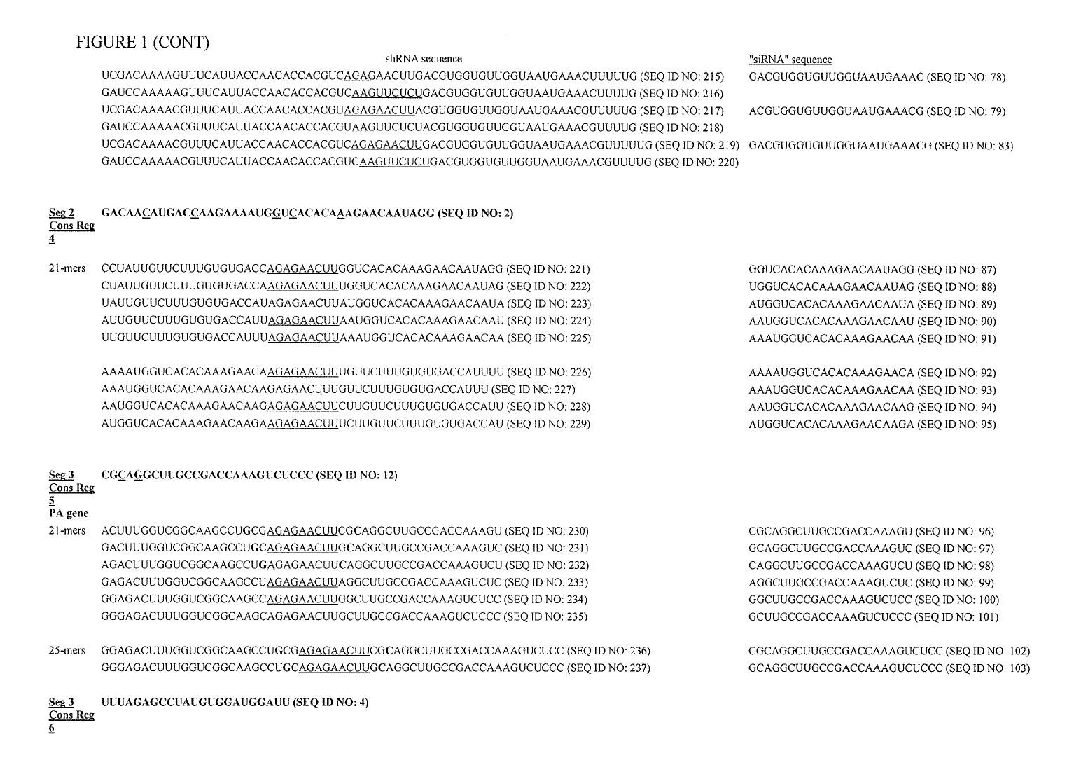

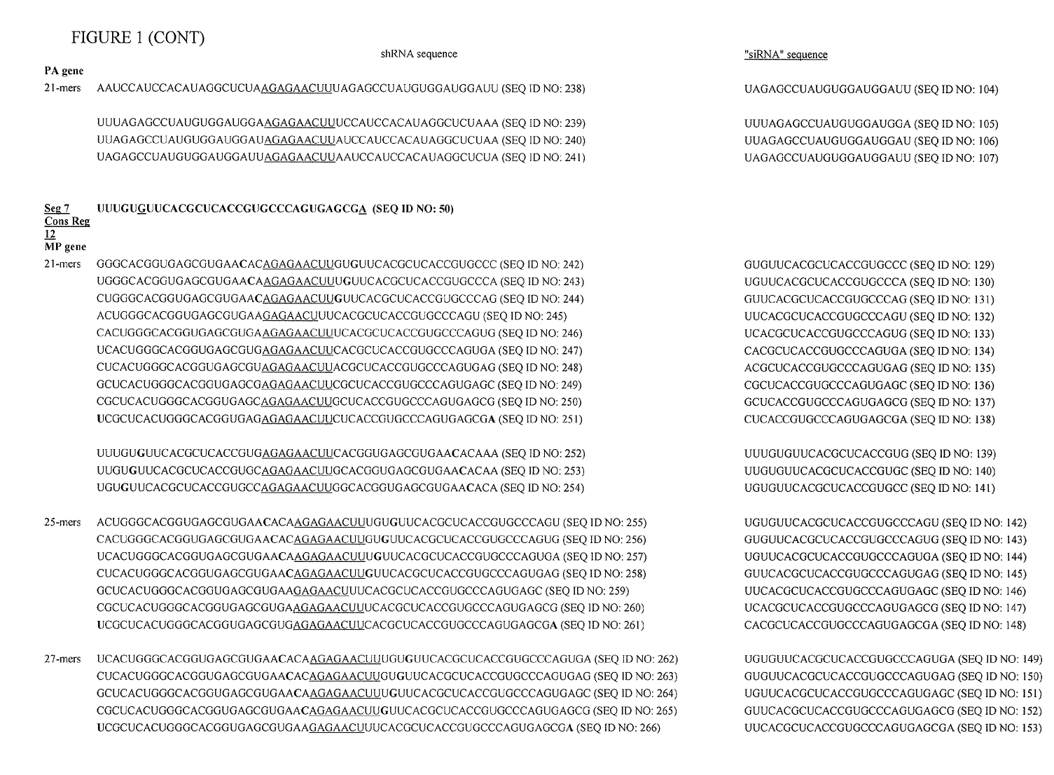

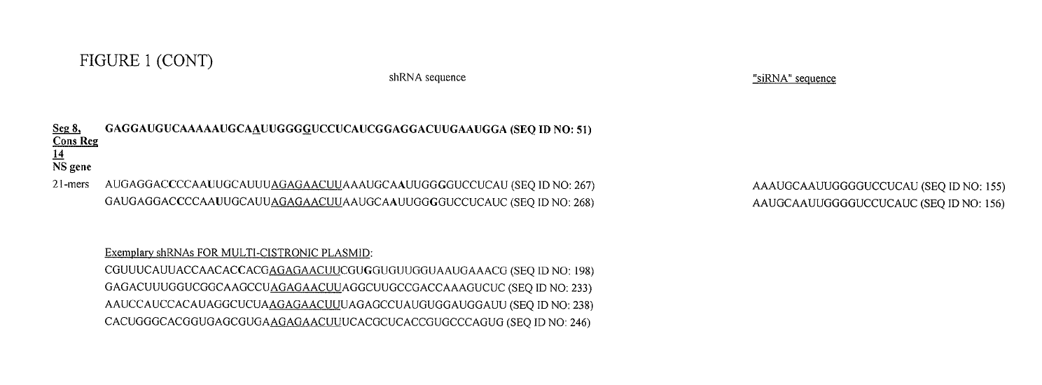

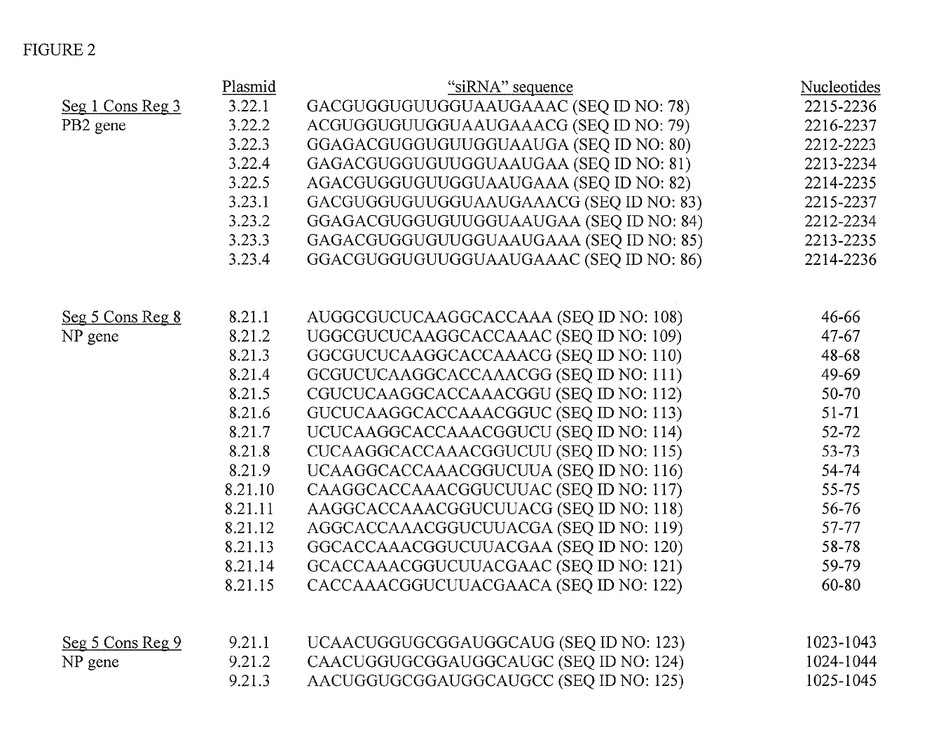

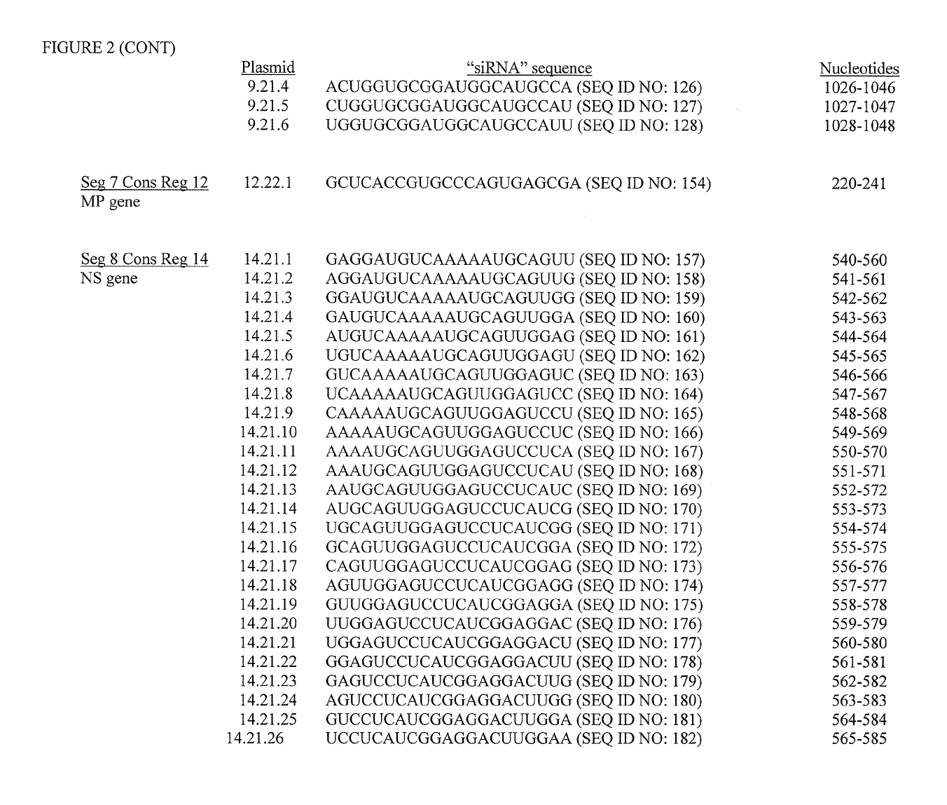

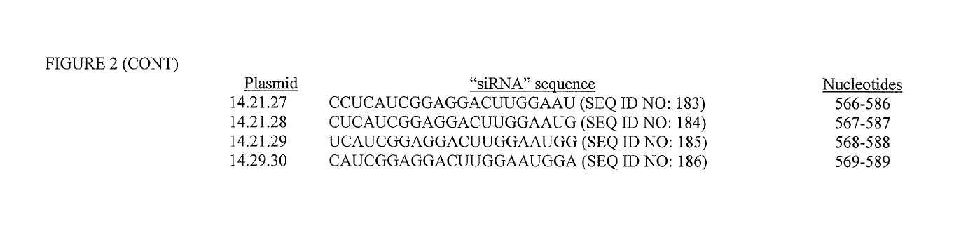

FIGS. 1 and 2 show exemplary siRNAs designed from the identified Influenza conserved regions.

In addition, FIG. 1 depicts shRNAs containing the siRNA sequences. The single stranded loop region is underlined.

FIG. 1 also shows an exemplary selection of shRNAs to be expressed, for example, from a multi-cistronic plasmid. These exemplary shRNAs correspond to Seg 1 Cons Reg 3, Seg 3 Cons Reg 5, Seg 3 Cons Reg 6, and Seg 7 Cons Reg 12.

### Example 3

### Inhibition of Influenza A Virus Replication Mediated by Expressed shRNAs

### Bioinformatics and Plasmid Design

A segment by segment comparison of all the influenza gene products between all the influenza A genomes present in GenBank version 150.0 was performed (Example 1). This comparison included more than 16,000 sequences from more than 200 different subtypes including human, avian and swine influenza. Plasmids expressing short-hairpin RNAs consisting of an antisense-loop-sense sequence against a conserved influenza mRNA target were constructed. Expression of these expressed short-hairpin RNAs is driven by a pol III promoter element. 26 plasmids were constructed which targeted a conserved region of the PB2 gene product located within nucleotides 2205-2237 in the PR/8strain. Plasmids were designed to express shRNAs of differing lengths against the targeted sequence, identified in FIG. 1 as Seg 1, Cons Region 3:

(SEQ ID NO: 10)

5′-GGGCAAGGAGACGUGGUGUUGGUAAUGAAACG-3′

As shown in FIG. 1: 12 plasmids expressing 21-mer shRNAs, 8 plasmids expressing 25-mer shRNAs, and 6 plasmids expressing 27-mer shRNAs were constructed to cover this entire 32 nucleotide conserved region of the PB2 gene. These plasmids were screened for their ability to inhibit influenza virus replication in a cell culture assay of virus infection using a hemagglutinin (HA) assay described below.

The plasmid designated 3.21.11, which expresses a 21-mer shRNA having the sequence,

5′-GUUUCAUUACCAACACCACGU-AGAGAACUU-ACGUGGUGUUGGUAAUGAAAC-3′ (SEQ ID NO: 197) (loop nucleotides are italicized), directed against the PB2 target sequence 5-ACGUGGUGUUGGUAAUGAAAC-3′ (SEQ ID NO: 62), was found to potently inhibit virus replication as determined by hemagglutinin assays and qRT-PCR of viral gene products. Interestingly, while the 3.21.11 plasmid expressing a shRNA including the ACGUGGUGUUGGUAAUGAAAC sequence (SEQ ID NO: 62) had a potent anti-viral effect, the 25-mer and 27-mer constructs which contained this sequence did not inhibit virus replication in the particular assay utilized.

### Experimental Design

Madin-Darby Canine Kidney (MDCK) cells (2×10⁶ in 0.1 mL) were transfected by electroporation using 2.0 ug of plasmids: NUC067 (negative control plasmid expressing a shRNA against a hepatitis B virus sequence) or 3.21.11

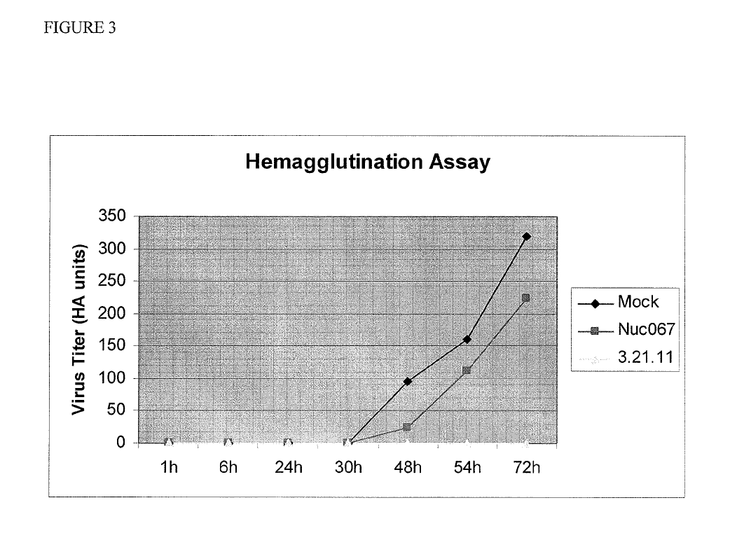

(plasmid expressing a shRNA directed against the PB2 gene product of influenza virus). Mock indicates cells that went through the electroporation procedure minus plasmid DNA. Cells were plated and ˜12 hours post electroporation cultures were infected with influenza A/PR/8/34 (H1N1) at a multiplicity of infection (MOI) of 0.01. Hemagglutinin assays were conducted on cell culture supernatants for each of the treated cell cultures at various time points. Briefly, twofold serial dilutions of cell culture supernatant samples were performed with PBS in V-shaped 96 well plates. Equal volumes of a 0.5% solution of chicken red blood cells (RBCs) in PBS were added to the wells and the plate incubated at 4° C. for 1 h. A lack of hemagglutination is indicated by the appearance of an RBC precipitate (RBC button). Hemagglutination titers were expressed as the inverse of the highest dilution of the sample able to agglutinate RBCs. The data shown in FIG. 3 are the average of 2 independent experiments run in duplicate.

### Results

As shown in FIG. 3, both the mock electroporated cells and cells transfected with a negative control eiRNA plasmid (NUC067) show increasing viral titers at 30 hours post-infection, which continue to increase over the time course of the experiment. Cells transfected with eiRNA 3.21.11 do not produce any detectable levels of virus in the cell culture supernatant using this assay for the complete time course of the experiment. Additionally, as H1N1 is cytopathic in MDCK cells the Mock and NUC067 cell culture monolayers were destroyed by the 72 h timepoint; by contrast the monolayer of the 3.21.11 transfected cells was intact (data not shown). These data indicate that within the limits of sensitivity of this assay the 3.21.11 expressing plasmid abolishes H1N1 replication in MDCK cells. qRT-PCR results (data not shown) indicate that this inhibition is mediated at the level of mRNA as several influenza gene products including the PB2, PB1, NS1, NP, and M messages were barely detectable compared to the mock treated influenza messages. These data indicate that 3.21.11 blocks H1N1 replication by inhibiting viral message expression. There was no evidence of escape mutants developing over the 72 hours that the experiment was run.

All publications, patents and patent applications discussed herein are incorporated herein by reference in their entireties. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated double-stranded polynucleotide consisting essentially of a conserved Influenza A sequence and its complementary sequence, said conserved Influenza A sequence being non-homologous to human sequences.

2. An isolated RNA molecule consisting of 19 or more contiguous nucleotides of a sequence selected from the group consisting of:
(SEQ ID NO: 1)
GGGCAAGGAGACGURGUGUUGGUAAUGAAACG,
(SEQ ID NO: 2)
GACAACAUGACCAAGAAAAUGGUCACACAAAGAACAAUAGG,
(SEQ ID NO: 3)
CGYAGGCUUGCCGACCAAAGUCUCCC,
(SEQ ID NO: 4)
UUUAGAGCCUAUGUGGAUGGAUU,
(SEQ ID NO: 5)
AUGGCGUCYCAAGGCACCAAACGRUCUUAUGARCA,
(SEQ ID NO: 6)
AGGCCCCCUCAAAGCCGARAUCGCDCAGAVACUUGAA,
(SEQ ID NO: 7)
UUUGURUUCACGCUCACCGUGCCCAGUGAGCGR,
(SEQ ID NO: 8)
AGAUGAUCUUCUUGAAAAUUUGCAGVCCUAYCAGAAACGRAUGGG,
and
(SEQ ID NO: 9)
GAGGAUGUCAAAAAUGCAAUUGGGGUCCUCAUCVDAGGRHUUGAA
UGGA,
wherein:
R is A or G,
Y is U or C,
D is A, G, or U,
V is A, G, or C, and
H is C, A, or U.

3. The RNA molecule of claim 2, wherein the RNA molecule consists of 19 or more contiguous nucleotides of a sequence selected from the group consisting of:
(SEQ ID NO: 10)
GGGCAAGGAGACGUGGUGUUGGUAAUGAAACG
(SEQ ID NO: 11)
GGGCAAGGAGACGUGAUGUUGGUAAUGAAACG
(SEQ ID NO: 12)
CGCAGGCUUGCCGACCAAAGUCUCCC
(SEQ ID NO: 13)
CGUAGGCUUGCCGACCAAAGUCUCCC
(SEQ ID NO: 14)
AUGGCGUCUCAAGGCACCAAACG,
(SEQ ID NO: 15)
AUGGCGUCCCAAGGCACCAAACG,
(SEQ ID NO: 16)
AGGCCCCCUCAAAGCCGAGAUCGC
(SEQ ID NO: 17)
AGGCCCCCUCAAAGCCGAAAUCGC
(SEQ ID NO: 18)
UUUGUAUUCACGCUCACCGUGCCCAGUGAGCGA,
(SEQ ID NO: 19)
UUUGUGUUCACGCUCACCGUGCCCAGUGAGCG,
(SEQ ID NO: 20)
UUCACGCUCACCGUGCCCAGUGAGCG
(SEQ ID NO: 21)
AGAUGAUCUUCUUGAAAAUUUGCAGGCCUA
(SEQ ID NO: 22)
AGAUGAUCUUCUUGAAAAUUUGCAGACCUA
(SEQ ID NO: 23), SEQ ID NOs: 52-186
GAGGAUGUCAAAAAUGCAAUUGGGGUCCUCAUCG.

4. The RNA molecule of claim 2, wherein said RNA molecule includes no more than one nucleotide designated as R, Y, D, V, or H.

5-7. (canceled)

8. The RNA molecule of claim 2, wherein said RNA molecule is hybridized to a substantially complementary RNA molecule.

9-26. (canceled)

27. An isolated double-stranded RNA comprising:
(1) a first region having 19 or more contiguous nucleotides of a sequence selected from the group consisting of:
(SEQ ID NO: 1)
GGGCAAGGAGACGURGUGUUGGUAAUGAAACG,
(SEQ ID NO: 2)
GACAACAUGACCAAGAAAAUGGUCACACAAAGAACAAUAGG,
(SEQ ID NO: 3)
CGYAGGCUUGCCGACCAAAGUCUCCC,
(SEQ ID NO: 4)
UUUAGAGCCUAUGUGGAUGGAUU,
(SEQ ID NO: 5)
AUGGCGUCYCAAGGCACCAAACGRUCUUAUGARCA,
(SEQ ID NO: 6)
AGGCCCCCUCAAAGCCGARAUCGCDCAGAVACUUGAA,
(SEQ ID NO: 7)
UUUGURUUCACGCUCACCGUGCCCAGUGAGCGR,
(SEQ ID NO: 8)
AGAUGAUCUUCUUGAAAAUUUGCAGVCCUAYCAGAAACGRAUGGG,
and
(SEQ ID NO: 9)
GAGGAUGUCAAAAAUGCAAUUGGGGUCCUCAUCVDAGGRHUUGAA
UGGA,
wherein:
R is A or G,
Y is U or C,
D is A, G, or U,
V is A, G, or C, and
H is C, A, or U; and
(2) a second region being substantially complementary to the first region.

28-32. (canceled)

33. The double-stranded RNA of claim 27, further comprising a single-stranded hairpin region connecting said first and second regions, and/or single-stranded 5′ and/or 3′ end(s).

34. The double-stranded RNA of claim 27, wherein the first region consists of 19 or more contiguous nucleotides of a sequence selected from the group consisting of:
(SEQ ID NO: 10)
GGGCAAGGAGACGUGGUGUUGGUAAUGAAACG
(SEQ ID NO: 11)
GGGCAAGGAGACGUGAUGUUGGUAAUGAAACG
(SEQ ID NO: 12)
CGCAGGCUUGCCGACCAAAGUCUCCC
(SEQ ID NO: 13)
CGUAGGCUUGCCGACCAAAGUCUCCC
(SEQ ID NO: 14)
AUGGCGUCUCAAGGCACCAAACG,
(SEQ ID NO: 15)
AUGGCGUCCCAAGGCACCAAACG,
(SEQ ID NO: 16)
AGGCCCCCUCAAAGCCGAGAUCGC
(SEQ ID NO: 17)
AGGCCCCCUCAAAGCCGAAAUCGC
(SEQ ID NO: 18)
UUUGUAUUCACGCUCACCGUGCCCAGUGAGCGA,
(SEQ ID NO: 19)
UUUGUGUUCACGCUCACCGUGCCCAGUGAGCG,
(SEQ ID NO: 20)
UUCACGCUCACCGUGCCCAGUGAGCG
(SEQ ID NO: 21)
AGAUGAUCUUCUUGAAAAUUUGCAGGCCUA
(SEQ ID NO: 22)
AGAUGAUCUUCUUGAAAAUUUGCAGACCUA
(SEQ ID NO:23), SEQ ID NOs: 52-186
GAGGAUGUCAAAAAUGCAAUUGGGGUCCUCAUCG.

35. The double-stranded RNA of claim 27, wherein the first region includes no more than one nucleotide designated as R, Y, D, V, or H.

36. (canceled)

37. (canceled)

38. A multi-target double-stranded RNA comprising:
(1) two or more segments each consisting of 19 or more contiguous nucleotides of a sequence selected from the group consisting of:
(SEQ ID NO: 1)
GGGCAAGGAGACGURGUGUUGGUAAUGAAACG,
(SEQ ID NO: 2)
GACAACAUGACCAAGAAAAUGGUCACACAAAGAACAAUAGG,
(SEQ ID NO: 3)
CGYAGGCUUGCCGACCAAAGUCUCCC,
(SEQ ID NO: 4)
UUUAGAGCCUAUGUGGAUGGAUU,
(SEQ ID NO: 5)
AUGGCGUCYCAAGGCACCAAACGRUCUUAUGARCA,
(SEQ ID NO: 6)
AGGCCCCCUCAAAGCCGARAUCGCDCAGAVACUUGAA,
(SEQ ID NO: 7)
UUUGURUUCACGCUCACCGUGCCCAGUGAGCGR,
(SEQ ID NO: 8)
AGAUGAUCUUCUUGAAAAUUUGCAGVCCUAYCAGAAACGRAUGGG,
and
(SEQ ID NO: 9)
GAGGAUGUCAAAAAUGCAAUUGGGGUCCUCAUCVDAGGRHUUGAA
UGGA,
wherein:
R is A or G,
Y is U or C,
D is A, G, or U,
V is A, G, or C, and
H is C, A, or U; and
(2) a substantially complementary region for each of said two or more segments,
wherein each of said two or more segments is connected to its complementary region through a single-stranded hairpin region.

39-43. (canceled)

44. An expression construct containing a DNA segment that encodes the RNA molecule of claim 2, said DNA segment being operably linked to one or more promoter(s).

45. The expression construct of claim 44, wherein said expression construct is a plasmid.

46. The expression construct of claim 45, wherein the plasmid encodes at least 2 said double-stranded RNA molecules.

47-54. (canceled)

55. A composition comprising:
(A) two or more RNA molecules each consisting of 19 or more contiguous nucleotides of a sequence selected from the group consisting of:
(SEQ ID NO: 1)
GGGCAAGGAGACGURGUGUUGGUAAUGAAACG,
(SEQ ID NO: 2)
GACAACAUGACCAAGAAAAUGGUCACACAAAGAACAAUAGG,
(SEQ ID NO: 3)
CGYAGGCUUGCCGACCAAAGUCUCCC,
(SEQ ID NO: 4)
UUUAGAGCCUAUGUGGAUGGAUU,
(SEQ ID NO: 5)
AUGGCGUCYCAAGGCACCAAACGRUCUUAUGARCA,
(SEQ ID NO: 6)
AGGCCCCCUCAAAGCCGARAUCGCDCAGAVACUUGAA,
(SEQ ID NO: 7)
UUUGURUUCACGCUCACCGUGCCCAGUGAGCGR,
(SEQ ID NO: 8)
AGAUGAUCUUCUUGAAAAUUUGCAGVCCUAYCAGAAACGRAUGGG,
and
(SEQ ID NO: 9)
GAGGAUGUCAAAAAUGCAAUUGGGGUCCUCAUCVDAGGRHUUGAA
UGGA,
wherein:
R is A or G,
Y is U or C,
D is A, G, or U,
V is A, G, or C, and
H is C, A, or U; and
(B) an RNA molecule complementary to each of said two or more RNA molecules, and
(C) a pharmaceutically acceptable carrier.

56. A composition comprising an expression vector encoding at least 2 RNA molecules of claim 2, and a pharmaceutically acceptable carrier.

57. (canceled)

58. A method of preventing Influenza A replication in a cell, comprising:
introducing the double-stranded RNA of claim 27, into a cell susceptible to Influenza A virus.

59. The method of claim 58, wherein said Influenza A is a human, swine or avian strain.

60. A method of reducing an Influenza A virus RNA, comprising: introducing the double-stranded RNA of claim 27, into a cell susceptible to Influenza A virus.

61. (canceled)

62. A method of treating a subject having an Influenza A viral infection, comprising introducing into said subject the double-stranded RNA of claim 27.

63. The method of claim 62, wherein or the double-stranded RNA, is introduced into the subject by administering an expression vector providing for expression in said subject of said double-stranded RNA molecule.

64. (canceled)

65. (canceled)


SEQUENCE LISTING (268 sequences)

SEQ ID NO: 1 - RNA, 32 nt, Influenza A virus
gggcaaggagacgurguguugguaaugaaacg

SEQ ID NO: 2 - RNA, 41 nt, Influenza A virus
gacaacaugaccaagaaaauggucacacaaagaacaauagg

SEQ ID NO: 3 - RNA, 26 nt, Influenza A virus
cgyaggcuugccgaccaaagucuccc

SEQ ID NO: 4 - RNA, 23 nt, Influenza A virus
uuuagagccuauguggauggauu

SEQ ID NO: 5 - RNA, 35 nt, Influenza A virus
auggcgucycaaggcaccaaacgrucuuaugarca

SEQ ID NO: 6 - RNA, 37 nt, Influenza A virus
aggcccccucaaagccgaraucgcdcagavacuugaa

SEQ ID NO: 7 - RNA, 33 nt, Influenza A virus
uuuguruucacgcucaccgugcccagugagcgr

SEQ ID NO: 8 - RNA, 45 nt, Influenza A virus
agaugaucuucuugaaaauuugcagvccuaycagaaacgrauggg

SEQ ID NO: 9 - RNA, 49 nt, Influenza A virus
gaggaugucaaaaaugcaauugggguccucaucvdaggrhuugaaugga

SEQ ID NO: 10 - RNA, 32 nt, Influenza A virus
gggcaaggagacgugguguugguaaugaaacg

SEQ ID NO: 11 - RNA, 32 nt, Influenza A virus
gggcaaggagacgugauguugguaaugaaacg

SEQ ID NO: 12 - RNA, 26 nt, Influenza A virus
cgcaggcuugccgaccaaagucuccc

SEQ ID NO: 13 - RNA, 26 nt, Influenza A virus
cguaggcuugccgaccaaagucuccc

SEQ ID NO: 14 - RNA, 23 nt, Influenza A virus
auggcgucucaaggcaccaaacg

SEQ ID NO: 15 - RNA, 23 nt, Influenza A virus
auggcgucccaaggcaccaaacg

SEQ ID NO: 16 - RNA, 24 nt, Influenza A virus
aggcccccucaaagccgagaucgc

SEQ ID NO: 17 - RNA, 24 nt, Influenza A virus
aggcccccucaaagccgaaaucgc

SEQ ID NO: 18 - RNA, 33 nt, Influenza A virus
uuuguauucacgcucaccgugcccagugagcga

SEQ ID NO: 19 - RNA, 32 nt, Influenza A virus
uuuguguucacgcucaccgugcccagugagcg

SEQ ID NO: 20 - RNA, 26 nt, Influenza A virus
uucacgcucaccgugcccagugagcg

SEQ ID NO: 21 - RNA, 30 nt, Influenza A virus
agaugaucuucuugaaaauuugcaggccua

SEQ ID NO: 22 - RNA, 30 nt, Influenza A virus
agaugaucuucuugaaaauuugcagaccua

SEQ ID NO: 23 - RNA, 34 nt, Influenza A virus
gaggaugucaaaaaugcaauugggguccucaucg

SEQ ID NO: 24 - DNA, 24 bp, Influenza A virus
  misc_feature (9)..(9): n is a, c, g, or t
atgachccnagcacngadatgtca

SEQ ID NO: 25 - DNA, 24 bp, Influenza A virus
atgactccaagcacagagatgtca

SEQ ID NO: 26 - DNA, 28 bp, Influenza A virus
gargtbagygaaacvcarggaacdgara

SEQ ID NO: 27 - DNA, 28 bp, Influenza A virus
gaggtcagtgaaacacagggaacagaga

SEQ ID NO: 28 - DNA, 32 bp, Influenza A virus
gggcaaggagacgtdgtgttggtaatgaaacg

SEQ ID NO: 29 - DNA, 32 bp, Influenza A virus
gggcaaggagacgtggtgttggtaatgaaacg

SEQ ID NO: 30 - DNA, 41 bp, Influenza A virus
gacaacatgaccaagaaaatggtcacacaaagaacaatagg

SEQ ID NO: 31 - DNA, 26 bp, Influenza A virus
cghaggcttgccgaccaaagtctccc

SEQ ID NO: 32 - DNA, 26 bp, Influenza A virus
cgcaggcttgccgaccaaagtctccc

SEQ ID NO: 33 - DNA, 23 bp, Influenza A virus
tttagagcctatgtggatggatt

SEQ ID NO: 34 - DNA, 23 bp, Influenza A virus
  misc_feature (9)..(9): n is a, c, g, or t
aghahtggnaatctaattgctcc

SEQ ID NO: 35 - DNA, 23 bp, Influenza A virus
agcaatgggaatctaattgctcc

SEQ ID NO: 36 - DNA, 35 bp, Influenza A virus
atggcgtcycaaggcaccaaacgrtcttatgarca

SEQ ID NO: 37 - DNA, 35 bp, Influenza A virus
atggcgtctcaaggcaccaaacgatcttatgaaca

SEQ ID NO: 38 - DNA, 26 bp, Influenza A virus
tcarytggtrtggatggcatgccatt

SEQ ID NO: 39 - DNA, 26 bp, Influenza A virus
tcagctggtgtggatggcatgccatt

SEQ ID NO: 40 - DNA, 21 bp, Influenza A virus
  misc_feature (6)..(6): n is a, c, g, or t
tccagntaydtntgcdcngga

SEQ ID NO: 41 - DNA, 21 bp, Influenza A virus
tccagttatgtgtgctcagga

SEQ ID NO: 42 - DNA, 37 bp, Influenza A virus
aggccccctcaaagccgaratcgcdcagavacttgaa

SEQ ID NO: 43 - DNA, 37 bp, Influenza A virus
aggccccctcaaagccgagatcgcgcagagacttgaa

SEQ ID NO: 44 - DNA, 33 bp, Influenza A virus
tttgtrttcacgctcaccgtgcccagtgagcgr

SEQ ID NO: 45 - DNA, 33 bp, Influenza A virus
tttgtgttcacgctcaccgtgcccagtgagcga

SEQ ID NO: 46 - DNA, 45 bp, Influenza A virus
agatgatcttcttgaaaatttgcagvcctaycagaaacgratggg

SEQ ID NO: 47 - DNA, 45 bp, Influenza A virus
agatgatcttcttgaaaatttgcaggcctaccagaaacgaatggg

SEQ ID NO: 48 - DNA, 49 bp, Influenza A virus
gaggatgtcaaaaatgcaattggggtcctcatcvdaggrhttgaatgga

SEQ ID NO: 49 - DNA, 49 bp, Influenza A virus
gaggatgtcaaaaatgcaattggggtcctcatcggaggacttgaatgga

SEQ ID NO: 50 - RNA, 33 nt, Influenza A virus
uuuguguucacgcucaccgugcccagugagcga

SEQ ID NO: 51 - RNA, 49 nt, Influenza A virus
gaggaugucaaaaaugcaauugggguccucaucggaggacuugaaugga

SEQ ID NO: 52 - RNA, 21 nt, Influenza A virus
gggcaaggagacgugguguug

SEQ ID NO: 53 - RNA, 21 nt, Influenza A virus
ggcaaggagacgugguguugg

SEQ ID NO: 54 - RNA, 21 nt, Influenza A virus
gcaaggagacgugguguuggu

SEQ ID NO: 55 - RNA, 21 nt, Influenza A virus
caaggagacgugguguuggua

SEQ ID NO: 56 - RNA, 21 nt, Influenza A virus
aaggagacgugguguugguaa

SEQ ID NO: 57 - RNA, 21 nt, Influenza A virus
aggagacgugguguugguaau

SEQ ID NO: 58 - RNA, 21 nt, Influenza A virus
ggagacgugguguugguaaug

SEQ ID NO: 59 - RNA, 21 nt, Influenza A virus
gagacgugguguugguaauga

SEQ ID NO: 60 - RNA, 21 nt, Influenza A virus
agacgugguguugguaaugaa

SEQ ID NO: 61 - RNA, 21 nt, Influenza A virus
gacgugguguugguaaugaaa

SEQ ID NO: 62 - RNA, 21 nt, Influenza A virus
acgugguguugguaaugaaac

SEQ ID NO: 63 - RNA, 21 nt, Influenza A virus
cgugguguugguaaugaaacg

SEQ ID NO: 64 - RNA, 25 nt, Influenza A virus
gggcaaggagacgugguguugguaa

SEQ ID NO: 65 - RNA, 25 nt, Influenza A virus
ggcaaggagacgugguguugguaau

SEQ ID NO: 66 - RNA, 25 nt, Influenza A virus
gcaaggagacgugguguugguaaug

SEQ ID NO: 67 - RNA, 25 nt, Influenza A virus
caaggagacgugguguugguaauga

SEQ ID NO: 68 - RNA, 25 nt, Influenza A virus
aaggagacgugguguugguaaugaa

SEQ ID NO: 69 - RNA, 25 nt, Influenza A virus
aggagacgugguguugguaaugaaa

SEQ ID NO: 70 - RNA, 25 nt, Influenza A virus
ggagacgugguguugguaaugaaac

SEQ ID NO: 71 - RNA, 25 nt, Influenza A virus
gagacgugguguugguaaugaaacg

SEQ ID NO: 72 - RNA, 27 nt, Influenza A virus
gggcaaggagacgugguguugguaaug

SEQ ID NO: 73 - RNA, 27 nt, Influenza A virus
ggcaaggagacgugguguugguaauga

SEQ ID NO: 74 - RNA, 27 nt, Influenza A virus
gcaaggagacgugguguugguaaugaa

SEQ ID NO: 75 - RNA, 27 nt, Influenza A virus
caaggagacgugguguugguaaugaaa

SEQ ID NO: 76 - RNA, 27 nt, Influenza A virus
aaggagacgugguguugguaaugaaac

SEQ ID NO: 77 - RNA, 27 nt, Influenza A virus
aggagacgugguguugguaaugaaacg

SEQ ID NO: 78 - RNA, 22 nt, Influenza A virus
gacgugguguugguaaugaaac

SEQ ID NO: 79 - RNA, 22 nt, Influenza A virus
acgugguguugguaaugaaacg

SEQ ID NO: 80 - RNA, 22 nt, Influenza A virus
ggagacgugguguugguaauga

SEQ ID NO: 81 - RNA, 22 nt, Influenza A virus
gagacgugguguugguaaugaa

SEQ ID NO: 82 - RNA, 22 nt, Influenza A virus
agacgugguguugguaaugaaa

SEQ ID NO: 83 - RNA, 23 nt, Influenza A virus
gacgugguguugguaaugaaacg

SEQ ID NO: 84 - RNA, 23 nt, Influenza A virus
ggagacgugguguugguaaugaa

SEQ ID NO: 85 - RNA, 23 nt, Influenza A virus
gagacgugguguugguaaugaaa

SEQ ID NO: 86 - RNA, 23 nt, Influenza A virus
ggacgugguguugguaaugaaac

SEQ ID NO: 87 - RNA, 21 nt, Influenza A virus
ggucacacaaagaacaauagg

SEQ ID NO: 88 - RNA, 21 nt, Influenza A virus
uggucacacaaagaacaauag

SEQ ID NO: 89 - RNA, 21 nt, Influenza A virus
auggucacacaaagaacaaua

SEQ ID NO: 90 - RNA, 21 nt, Influenza A virus
aauggucacacaaagaacaau

SEQ ID NO: 91 - RNA, 21 nt, Influenza A virus
aaauggucacacaaagaacaa

SEQ ID NO: 92 - RNA, 21 nt, Influenza A virus
aaaauggucacacaaagaaca

SEQ ID NO: 93 - RNA, 21 nt, Influenza A virus
aaauggucacacaaagaacaa

SEQ ID NO: 94 - RNA, 21 nt, Influenza A virus
aauggucacacaaagaacaag

SEQ ID NO: 95 - RNA, 21 nt, Influenza A virus
auggucacacaaagaacaaga

SEQ ID NO: 96 - RNA, 21 nt, Influenza A virus
cgcaggcuugccgaccaaagu

SEQ ID NO: 97 - RNA, 21 nt, Influenza A virus
gcaggcuugccgaccaaaguc

SEQ ID NO: 98 - RNA, 21 nt, Influenza A virus
caggcuugccgaccaaagucu

SEQ ID NO: 99 - RNA, 21 nt, Influenza A virus
aggcuugccgaccaaagucuc

SEQ ID NO: 100 - RNA, 21 nt, Influenza A virus
ggcuugccgaccaaagucucc

SEQ ID NO: 101 - RNA, 21 nt, Influenza A virus
gcuugccgaccaaagucuccc

SEQ ID NO: 102 - RNA, 25 nt, Influenza A virus
cgcaggcuugccgaccaaagucucc

SEQ ID NO: 103 - RNA, 25 nt, Influenza A virus
gcaggcuugccgaccaaagucuccc

SEQ ID NO: 104 - RNA, 21 nt, Influenza A virus
uagagccuauguggauggauu

SEQ ID NO: 105 - RNA, 21 nt, Influenza A virus
uuuagagccuauguggaugga

SEQ ID NO: 106 - RNA, 21 nt, Influenza A virus
uuagagccuauguggauggau

SEQ ID NO: 107 - RNA, 21 nt, Influenza A virus
uagagccuauguggauggauu

SEQ ID NO: 108 - RNA, 21 nt, Influenza A virus
auggcgucucaaggcaccaaa

SEQ ID NO: 109 - RNA, 21 nt, Influenza A virus
uggcgucucaaggcaccaaac

SEQ ID NO: 110 - RNA, 21 nt, Influenza A virus
ggcgucucaaggcaccaaacg

SEQ ID NO: 111 - RNA, 21 nt, Influenza A virus
gcgucucaaggcaccaaacgg

SEQ ID NO: 112 - RNA, 21 nt, Influenza A virus
cgucucaaggcaccaaacggu

SEQ ID NO: 113 - RNA, 21 nt, Influenza A virus
gucucaaggcaccaaacgguc

SEQ ID NO: 114 - RNA, 21 nt, Influenza A virus
ucucaaggcaccaaacggucu

SEQ ID NO: 115 - RNA, 21 nt, Influenza A virus
cucaaggcaccaaacggucuu

SEQ ID NO: 116 - RNA, 21 nt, Influenza A virus
ucaaggcaccaaacggucuua

SEQ ID NO: 117 - RNA, 21 nt, Influenza A virus
caaggcaccaaacggucuuac

SEQ ID NO: 118 - RNA, 21 nt, Influenza A virus
aaggcaccaaacggucuuacg

SEQ ID NO: 119 - RNA, 21 nt, Influenza A virus
aggcaccaaacggucuuacga

SEQ ID NO: 120 - RNA, 21 nt, Influenza A virus
ggcaccaaacggucuuacgaa

SEQ ID NO: 121 - RNA, 21 nt, Influenza A virus
gcaccaaacggucuuacgaac

SEQ ID NO: 122 - RNA, 21 nt, Influenza A virus
caccaaacggucuuacgaaca

SEQ ID NO: 123 - RNA, 21 nt, Influenza A virus
ucaacuggugcggauggcaug

SEQ ID NO: 124 - RNA, 21 nt, Influenza A virus
caacuggugcggauggcaugc

SEQ ID NO: 125 - RNA, 21 nt, Influenza A virus
aacuggugcggauggcaugcc

SEQ ID NO: 126 - RNA, 21 nt, Influenza A virus
acuggugcggauggcaugcca

SEQ ID NO: 127 - RNA, 21 nt, Influenza A virus
cuggugcggauggcaugccau

SEQ ID NO: 128 - RNA, 21 nt, Influenza A virus
uggugcggauggcaugccauu

SEQ ID NO: 129 - RNA, 21 nt, Influenza A virus
guguucacgcucaccgugccc

SEQ ID NO: 130 - RNA, 21 nt, Influenza A virus
uguucacgcucaccgugccca

SEQ ID NO: 131 - RNA, 21 nt, Influenza A virus
guucacgcucaccgugcccag

SEQ ID NO: 132 - RNA, 21 nt, Influenza A virus
uucacgcucaccgugcccagu

SEQ ID NO: 133 - RNA, 21 nt, Influenza A virus
ucacgcucaccgugcccagug

SEQ ID NO: 134 - RNA, 21 nt, Influenza A virus
cacgcucaccgugcccaguga

SEQ ID NO: 135 - RNA, 21 nt, Influenza A virus
acgcucaccgugcccagugag

SEQ ID NO: 136 - RNA, 21 nt, Influenza A virus
cgcucaccgugcccagugagc

SEQ ID NO: 137 - RNA, 21 nt, Influenza A virus
gcucaccgugcccagugagcg

SEQ ID NO: 138 - RNA, 21 nt, Influenza A virus
cucaccgugcccagugagcga

SEQ ID NO: 139 - RNA, 21 nt, Influenza A virus
uuuguguucacgcucaccgug

SEQ ID NO: 140 - RNA, 21 nt, Influenza A virus
uuguguucacgcucaccgugc

SEQ ID NO: 141 - RNA, 21 nt, Influenza A virus
uguguucacgcucaccgugcc

SEQ ID NO: 142 - RNA, 25 nt, Influenza A virus
uguguucacgcucaccgugcccagu

SEQ ID NO: 143 - RNA, 25 nt, Influenza A virus
guguucacgcucaccgugcccagug

SEQ ID NO: 144 - RNA, 25 nt, Influenza A virus
uguucacgcucaccgugcccaguga

SEQ ID NO: 145 - RNA, 25 nt, Influenza A virus
guucacgcucaccgugcccagugag

SEQ ID NO: 146 - RNA, 25 nt, Influenza A virus
uucacgcucaccgugcccagugagc

SEQ ID NO: 147 - RNA, 25 nt, Influenza A virus
ucacgcucaccgugcccagugagcg

SEQ ID NO: 148 - RNA, 25 nt, Influenza A virus
cacgcucaccgugcccagugagcga

SEQ ID NO: 149 - RNA, 27 nt, Influenza A virus
uguguucacgcucaccgugcccaguga

SEQ ID NO: 150 - RNA, 27 nt, Influenza A virus
guguucacgcucaccgugcccagugag

SEQ ID NO: 151 - RNA, 27 nt, Influenza A virus
uguucacgcucaccgugcccagugagc

SEQ ID NO: 152 - RNA, 27 nt, Influenza A virus
guucacgcucaccgugcccagugagcg

SEQ ID NO: 153 - RNA, 27 nt, Influenza A virus
uucacgcucaccgugcccagugagcga

SEQ ID NO: 154 - RNA, 22 nt, Influenza A virus
gcucaccgugcccagugagcga

SEQ ID NO: 155 - RNA, 21 nt, Influenza A virus
aaaugcaauugggguccucau

SEQ ID NO: 156 - RNA, 21 nt, Influenza A virus
aaugcaauugggguccucauc

SEQ ID NO: 157 - RNA, 21 nt, Influenza A virus
gaggaugucaaaaaugcaguu

SEQ ID NO: 158 - RNA, 21 nt, Influenza A virus
aggaugucaaaaaugcaguug

SEQ ID NO: 159 - RNA, 21 nt, Influenza A virus
ggaugucaaaaaugcaguugg

SEQ ID NO: 160 - RNA, 21 nt, Influenza A virus
gaugucaaaaaugcaguugga

SEQ ID NO: 161 - RNA, 21 nt, Influenza A virus
augucaaaaaugcaguuggag

SEQ ID NO: 162 - RNA, 21 nt, Influenza A virus
ugucaaaaaugcaguuggagu

SEQ ID NO: 163 - RNA, 21 nt, Influenza A virus
gucaaaaaugcaguuggaguc

SEQ ID NO: 164 - RNA, 21 nt, Influenza A virus
ucaaaaaugcaguuggagucc

SEQ ID NO: 165 - RNA, 21 nt, Influenza A virus
caaaaaugcaguuggaguccu

SEQ ID NO: 166 - RNA, 21 nt, Influenza A virus
aaaaaugcaguuggaguccuc

SEQ ID NO: 167 - RNA, 21 nt, Influenza A virus
aaaaugcaguuggaguccuca

SEQ ID NO: 168 - RNA, 21 nt, Influenza A virus
aaaugcaguuggaguccucau

SEQ ID NO: 169 - RNA, 21 nt, Influenza A virus
aaugcaguuggaguccucauc

SEQ ID NO: 170 - RNA, 21 nt, Influenza A virus
augcaguuggaguccucaucg

SEQ ID NO: 171 - RNA, 21 nt, Influenza A virus
ugcaguuggaguccucaucgg

SEQ ID NO: 172 - RNA, 21 nt, Influenza A virus
gcaguuggaguccucaucgga

SEQ ID NO: 173 - RNA, 21 nt, Influenza A virus
caguuggaguccucaucggag

SEQ ID NO: 174 - RNA, 21 nt, Influenza A virus
aguuggaguccucaucggagg

SEQ ID NO: 175 - RNA, 21 nt, Influenza A virus
guuggaguccucaucggagga

SEQ ID NO: 176 - RNA, 21 nt, Influenza A virus
uuggaguccucaucggaggac

SEQ ID NO: 177 - RNA, 21 nt, Influenza A virus
uggaguccucaucggaggacu

SEQ ID NO: 178 - RNA, 21 nt, Influenza A virus
ggaguccucaucggaggacuu

SEQ ID NO: 179 - RNA, 21 nt, Influenza A virus
gaguccucaucggaggacuug

SEQ ID NO: 180 - RNA, 21 nt, Influenza A virus
aguccucaucggaggacuugg

SEQ ID NO: 181 - RNA, 21 nt, Influenza A virus
guccucaucggaggacuugga

SEQ ID NO: 182 - RNA, 21 nt, Influenza A virus
uccucaucggaggacuuggaa

SEQ ID NO: 183 - RNA, 21 nt, Influenza A virus
ccucaucggaggacuuggaau

SEQ ID NO: 184 - RNA, 21 nt, Influenza A virus
cucaucggaggacuuggaaug

SEQ ID NO: 185 - RNA, 21 nt, Influenza A virus
ucaucggaggacuuggaaugg

SEQ ID NO: 186 - RNA, 21 nt, Influenza A virus
caucggaggacuuggaaugga

SEQ ID NO: 187 - RNA, 51 nt, Influenza A virus
caacaccacgucuccuugcccagagaacuugggcaaggagacgugguguug

SEQ ID NO: 188 - RNA, 51 nt, Influenza A virus
ccaacaccacgucuccuugccagagaacuuggcaaggagacgugguguugg

SEQ ID NO: 189 - RNA, 51 nt, Influenza A virus
accaacaccacgucuccuugcagagaacuugcaaggagacgugguguuggu

SEQ ID NO: 190 - RNA, 51 nt, Influenza A virus
uaccaacaccacgucuccuugagagaacuucaaggagacgugguguuggua

SEQ ID NO: 191 - RNA, 51 nt, Influenza A virus
uuaccaacaccacgucuccuuagagaacuuaaggagacgugguguugguaa

SEQ ID NO: 192 - RNA, 51 nt, Influenza A virus
auuaccaacaccacgucuccuagagaacuuaggagacgugguguugguaau

SEQ ID NO: 193 - RNA, 51 nt, Influenza A virus
cauuaccaacaccacgucuccagagaacuuggagacgugguguugguaaug

SEQ ID NO: 194 - RNA, 51 nt, Influenza A virus
ucauuaccaacaccacgucucagagaacuugagacgugguguugguaauga

SEQ ID NO: 195 - RNA, 51 nt, Influenza A virus
uucauuaccaacaccacgucuagagaacuuagacgugguguugguaaugaa

SEQ ID NO: 196 - RNA, 51 nt, Influenza A virus
uuucauuaccaacaccacgucagagaacuugacgugguguugguaaugaaa

SEQ ID NO: 197 - RNA, 51 nt, Influenza A virus
guuucauuaccaacaccacguagagaacuuacgugguguugguaaugaaac

SEQ ID NO: 198 - RNA, 51 nt, Influenza A virus
cguuucauuaccaacaccacgagagaacuucgugguguugguaaugaaacg

SEQ ID NO: 199 - RNA, 59 nt, Influenza A virus
uuaccaacaccacgucuccuugcccagagaacuugggcaaggagacgugguguugguaa

SEQ ID NO: 200 - RNA, 59 nt, Influenza A virus
auuaccaacaccacgucuccuugccagagaacuuggcaaggagacgugguguugguaau

SEQ ID NO: 201 - RNA, 59 nt, Influenza A virus
cauuaccaacaccacgucuccuugcagagaacuugcaaggagacgugguguugguaaug

SEQ ID NO: 202 - RNA, 59 nt, Influenza A virus
ucauuaccaacaccacgucuccuugagagaacuucaaggagacgugguguugguaauga

SEQ ID NO: 203 - RNA, 59 nt, Influenza A virus
uucauuaccaacaccacgucuccuuagagaacuuaaggagacgugguguugguaaugaa

SEQ ID NO: 204 - RNA, 59 nt, Influenza A virus
uuucauuaccaacaccacgucuccuagagaacuuaggagacgugguguugguaaugaaa

SEQ ID NO: 205 - RNA, 59 nt, Influenza A virus
guuucauuaccaacaccacgucuccagagaacuuggagacgugguguugguaaugaaac

SEQ ID NO: 206 - RNA, 59 nt, Influenza A virus
cguuucauuaccaacaccacgucucagagaacuugagacgugguguugguaaugaaacg

SEQ ID NO: 207 - RNA, 63 nt, Influenza A virus
cauuaccaacaccacgucuccuugcccagagaacuugggcaaggagacgugguguugguaaug

SEQ ID NO: 208 - RNA, 63 nt, Influenza A virus
ucauuaccaacaccacgucuccuugccagagaacuuggcaaggagacgugguguugguaauga

SEQ ID NO: 209 - RNA, 63 nt, Influenza A virus
uucauuaccaacaccacgucuccuugcagagaacuugcaaggagacgugguguugguaaugaa

SEQ ID NO: 210 - RNA, 63 nt, Influenza A virus
uuucauuaccaacaccacgucuccuugagagaacuucaaggagacgugguguugguaaugaaa

SEQ ID NO: 211 - RNA, 63 nt, Influenza A virus
guuucauuaccaacaccacgucuccuuagagaacuuaaggagacgugguguugguaaugaaac

SEQ ID NO: 212 - RNA, 63 nt, Influenza A virus
cguuucauuaccaacaccacgucuccuagagaacuuaggagacgugguguugguaaugaaacg

SEQ ID NO: 213 - RNA, 66 nt, Influenza A virus
ucgacaaaaguuucauuaccaacaccacguagagaacuuacgugguguugguaaugaaacuuuuug

SEQ ID NO: 214 - RNA, 66 nt, Influenza A virus
gauccaaaaauuucauuaccaacaccacgucaaguucucuacgugguguugguaaugaaacuuuug

SEQ ID NO: 215 - RNA, 68 nt, Influenza A virus
ucgacaaaaguuucauuaccaacaccacgucagagaacuugacgugguguugguaaugaaacuuuuug

SEQ ID NO: 216 - RNA, 68 nt, Influenza A virus
gauccaaaaaguuucauuaccaacaccacgucaaguucucugacgugguguugguaaugaaacuuuug

SEQ ID NO: 217 - RNA, 68 nt, Influenza A virus
ucgacaaaacguuucauuaccaacaccacguagagaacuuacgugguguugguaaugaaacguuuuug

SEQ ID NO: 218 - RNA, 68 nt, Influenza A virus
gauccaaaaacguuucauuaccaacaccacguaaguucucuacgugguguugguaaugaaacguuuug

SEQ ID NO: 219 - RNA, 70 nt, Influenza A virus
ucgacaaaacguuucauuaccaacaccacgucagagaacuugacgugguguugguaaugaaacguuuuug

SEQ ID NO: 220 - RNA, 70 nt, Influenza A virus
gauccaaaaacguuucauuaccaacaccacgucaaguucucugacgugguguugguaaugaaacguuuug

SEQ ID NO: 221 - RNA, 51 nt, Influenza A virus
ccuauuguucuuugugugaccagagaacuuggucacacaaagaacaauagg

SEQ ID NO: 222 - RNA, 51 nt, Influenza A virus
cuauuguucuuugugugaccaagagaacuuuggucacacaaagaacaauag

SEQ ID NO: 223 - RNA, 51 nt, Influenza A virus
uauuguucuuugugugaccauagagaacuuauggucacacaaagaacaaua

SEQ ID NO: 224 - RNA, 51 nt, Influenza A virus
auuguucuuugugugaccauuagagaacuuaauggucacacaaagaacaau

SEQ ID NO: 225 - RNA, 51 nt, Influenza A virus
uuguucuuugugugaccauuuagagaacuuaaauggucacacaaagaacaa

SEQ ID NO: 226 - RNA, 51 nt, Influenza A virus
aaaauggucacacaaagaacaagagaacuuuguucuuugugugaccauuuu

SEQ ID NO: 227 - RNA, 49 nt, Influenza A virus
aaauggucacacaaagaacaagagaacuuuguucuuugugugaccauuu

SEQ ID NO: 228 - RNA, 51 nt, Influenza A virus
aauggucacacaaagaacaagagagaacuucuuguucuuugugugaccauu

SEQ ID NO: 229 - RNA, 51 nt, Influenza A virus
auggucacacaaagaacaagaagagaacuuucuuguucuuugugugaccau

SEQ ID NO: 230 - RNA, 51 nt, Influenza A virus
acuuuggucggcaagccugcgagagaacuucgcaggcuugccgaccaaagu

SEQ ID NO: 231 - RNA, 51 nt, Influenza A virus
gacuuuggucggcaagccugcagagaacuugcaggcuugccgaccaaaguc

SEQ ID NO: 232 - RNA, 51 nt, Influenza A virus
agacuuuggucggcaagccugagagaacuucaggcuugccgaccaaagucu

SEQ ID NO: 233 - RNA, 51 nt, Influenza A virus
gagacuuuggucggcaagccuagagaacuuaggcuugccgaccaaagucuc

SEQ ID NO: 234 - RNA, 51 nt, Influenza A virus
ggagacuuuggucggcaagccagagaacuuggcuugccgaccaaagucucc

SEQ ID NO: 235 - RNA, 51 nt, Influenza A virus
gggagacuuuggucggcaagcagagaacuugcuugccgaccaaagucuccc

SEQ ID NO: 236 - RNA, 59 nt, Influenza A virus
ggagacuuuggucggcaagccugcgagagaacuucgcaggcuugccgaccaaagucucc

SEQ ID NO: 237 - RNA, 59 nt, Influenza A virus
gggagacuuuggucggcaagccugcagagaacuugcaggcuugccgaccaaagucuccc

SEQ ID NO: 238 - RNA, 51 nt, Influenza A virus
aauccauccacauaggcucuaagagaacuuuagagccuauguggauggauu

SEQ ID NO: 239 - RNA, 51 nt, Influenza A virus
uuuagagccuauguggauggaagagaacuuuccauccacauaggcucuaaa

SEQ ID NO: 240 - RNA, 51 nt, Influenza A virus
uuagagccuauguggauggauagagaacuuauccauccacauaggcucuaa

SEQ ID NO: 241 - RNA, 51 nt, Influenza A virus
uagagccuauguggauggauuagagaacuuaauccauccacauaggcucua

SEQ ID NO: 242 - RNA, 51 nt, Influenza A virus
gggcacggugagcgugaacacagagaacuuguguucacgcucaccgugccc

SEQ ID NO: 243 - RNA, 51 nt, Influenza A virus
ugggcacggugagcgugaacaagagaacuuuguucacgcucaccgugccca

SEQ ID NO: 244 - RNA, 51 nt, Influenza A virus
cugggcacggugagcgugaacagagaacuuguucacgcucaccgugcccag

SEQ ID NO: 245 - RNA, 49 nt, Influenza A virus
acugggcacggugagcgugaagagaacuuucacgcucaccgugcccagu

SEQ ID NO: 246 - RNA, 51 nt, Influenza A virus
cacugggcacggugagcgugaagagaacuuucacgcucaccgugcccagug

SEQ ID NO: 247 - RNA, 51 nt, Influenza A virus
ucacugggcacggugagcgugagagaacuucacgcucaccgugcccaguga

SEQ ID NO: 248 - RNA, 51 nt, Influenza A virus
cucacugggcacggugagcguagagaacuuacgcucaccgugcccagugag

SEQ ID NO: 249 - RNA, 51 nt, Influenza A virus
gcucacugggcacggugagcgagagaacuucgcucaccgugcccagugagc

SEQ ID NO: 250 - RNA, 51 nt, Influenza A virus
cgcucacugggcacggugagcagagaacuugcucaccgugcccagugagcg

SEQ ID NO: 251 - RNA, 51 nt, Influenza A virus
ucgcucacugggcacggugagagagaacuucucaccgugcccagugagcga

SEQ ID NO: 252 - RNA, 51 nt, Influenza A virus
uuuguguucacgcucaccgugagagaacuucacggugagcgugaacacaaa

SEQ ID NO: 253 - RNA, 51 nt, Influenza A virus
uuguguucacgcucaccgugcagagaacuugcacggugagcgugaacacaa

SEQ ID NO: 254 - RNA, 51 nt, Influenza A virus
uguguucacgcucaccgugccagagaacuuggcacggugagcgugaacaca

SEQ ID NO: 255 - RNA, 59 nt, Influenza A virus
acugggcacggugagcgugaacacaagagaacuuuguguucacgcucaccgugcccagu

SEQ ID NO: 256 - RNA, 59 nt, Influenza A virus
cacugggcacggugagcgugaacacagagaacuuguguucacgcucaccgugcccagug

SEQ ID NO: 257 - RNA, 59 nt, Influenza A virus
ucacugggcacggugagcgugaacaagagaacuuuguucacgcucaccgugcccaguga

SEQ ID NO: 258 - RNA, 59 nt, Influenza A virus
cucacugggcacggugagcgugaacagagaacuuguucacgcucaccgugcccagugag

SEQ ID NO: 259 - RNA, 57 nt, Influenza A virus
gcucacugggcacggugagcgugaagagaacuuucacgcucaccgugcccagugagc

SEQ ID NO: 260 - RNA, 59 nt, Influenza A virus
cgcucacugggcacggugagcgugaagagaacuuucacgcucaccgugcccagugagcg

SEQ ID NO: 261 - RNA, 59 nt, Influenza A virus
ucgcucacugggcacggugagcgugagagaacuucacgcucaccgugcccagugagcga

SEQ ID NO: 262 - RNA, 63 nt, Influenza A virus
ucacugggcacggugagcgugaacacaagagaacuuuguguucacgcucaccgugcccaguga

SEQ ID NO: 263 - RNA, 63 nt, Influenza A virus
cucacugggcacggugagcgugaacacagagaacuuguguucacgcucaccgugcccagugag

SEQ ID NO: 264 - RNA, 63 nt, Influenza A virus
gcucacugggcacggugagcgugaacaagagaacuuuguucacgcucaccgugcccagugagc

SEQ ID NO: 265 - RNA, 63 nt, Influenza A virus
cgcucacugggcacggugagcgugaacagagaacuuguucacgcucaccgugcccagugagcg

SEQ ID NO: 266 - RNA, 61 nt, Influenza A virus
ucgcucacugggcacggugagcgugaagagaacuuucacgcucaccgugcccagugagcga

SEQ ID NO: 267 - RNA, 51 nt, Influenza A virus
augaggaccccaauugcauuuagagaacuuaaaugcaauugggguccucau

SEQ ID NO: 268 - RNA, 51 nt, Influenza A virus
gaugaggaccccaauugcauuagagaacuuaaugcaauugggguccucauc